(19) United States
(12) Patent Application Publication
(10) Pub. No.: US 2010/0016360 A1
(43) Pub. Date: Jan. 21, 2010

(54) ALPHA7 NICOTINIC ACETYLCHOLINE RECEPTOR INHIBITORS

(75) Inventors: Simon N. Haydar, Newtown, PA (US); Chiara Ghiron, Asciano (IT); Michela Valacchi, Firenze (IT); Ugo Zanelli, Pisa (IT)

Correspondence Address: CHOATE, HALL & STEWART LLP, TWO INTERNATIONAL PLACE, BOSTON, MA, 02110, US

(73) Assignees: WYETH, Madison, NJ (US); SIENA BIOTECH S.P.A., Siena (IT)

(21) Appl. No.: 12/504,547

(22) Filed: Jul. 16, 2009

Related U.S. Application Data
(60) Provisional application No. 61/081,198, filed on Jul. 16, 2008.

(51) Int. Cl. A61K 31/4545; C07D 401/12; A61P 25/18; A61P 25/28

(52) U.S. Cl. 514/318; 546/194

(57) ABSTRACT

The present invention provides compounds and compositions, methods of making them, and methods of using them to modulate α7 nicotinic acetylcholine receptors and/or to treat any of a variety of disorders, diseases, and conditions. Provided compounds can affect, among other things, neurological, psychiatric and/or inflammatory system.

### CROSS REFERENCE TO RELATED APPLICATIONS

This application claims priority to U.S. provisional patent application Ser. No. 61/081,198, filed Jul. 16, 2008, the entirety of which is hereby incorporated herein by reference.

### FIELD OF THE INVENTION

The present invention relates to compounds with α7 nicotinic acetylcholine receptor (α7 nAChR) agonistic activity, syntheses thereof, and intermediates thereto.

### BACKGROUND OF THE INVENTION

Agents that bind to nicotinic acetylcholine receptors have been indicated as useful in the treatment and/or prophylaxis of various diseases and conditions, particularly psychotic diseases, neurodegenerative diseases involving a dysfunction of the cholinergic system, and conditions of memory and/or cognition impairment, including for example, schizophrenia, anxiety, mania, depression, manic depression, Tourette's syndrome, Parkinson's disease, Huntington's disease, cognitive disorders (such as Alzheimer's disease, Lewy Body Dementia, Amyotrophic Lateral Sclerosis, memory impairment, memory loss, cognition deficit, attention deficit, Attention Deficit Hyperactivity Disorder), and other uses such as treatment of nicotine addiction, inducing smoking cessation, treating pain (e.g. analgesic use), providing neuroprotection, and treating jetlag. See for example WO 97/30998; WO 99/03850; WO 00/42044; WO 01/36417; Holladay et al., J. Med. Chem., 40:26, 4169-94 (1997); Schmitt et al., Annual Reports Med. Chem., Chapter 5, 41-51 (2000); Stevens et al., Psychopharmatology, (1998) 136: 320-27; and Shytle et al., Molecular Psychiatry, (2002), 7, pp. 525-535.

Different heterocyclic compounds carrying a basic nitrogen and exhibiting nicotinic and muscarinic acetylcholine receptor affinity or claimed for use in Alzheimer's disease have been described, e.g. 1H-pyrazole and pyrrole-azabicyclic compounds (WO2004013137); nicotinic acetylcholine agonists (WO2004039366); ureido-pyrazole derivatives (WO0112188); oxadiazole derivatives having acetylcholinesterase-inhibitory activity and muscarinic agonist activity (WO9313083); pyrazole-3-carboxylic acid amide derivatives as pharmaceutical compounds (WO2006077428); arylpiperidines (WO2004006924); ureidoalkylpiperidines (U.S. Pat. No. 6,605,623); compounds with activity on muscarinic receptors (WO9950247). In addition, modulators of alpha7 nicotinic acetylcholine receptor are disclosed in WO06008133, in the name of the same applicant.

### SUMMARY OF THE INVENTION

The present disclosure encompasses the recognition that compounds acting as full or partial agonists at the α7 nicotinic acetylcholine receptor (α7 nAChR) are useful for the treatment of diseases such as neurological, neurodegenerative, psychiatric, cognitive, immunological, inflammatory, metabolic, addiction, nociceptive, and sexual disorders, in particular Alzheimer's disease, schizophrenia, and/or others. Such compounds include those of formula I:

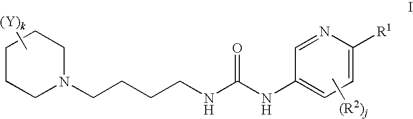

or a pharmaceutically acceptable salt thereof, wherein each of j, k, R¹, R², and Y is as defined herein.

### BRIEF DESCRIPTION OF THE DRAWINGS

FIG. 1 depicts the X-ray diffraction pattern of compound I-4 hydrochloride monohydrate.

FIG. 2 depicts the TGA scan of compound I-4 hydrochloride monohydrate.

FIG. 3 depicts the DSC scan of compound I-4 hydrochloride monohydrate.

FIG. 4 depicts the X-ray diffraction pattern of compound I-4 hydrochloride anhydrous.

FIG. 5 depicts the TGA scan of compound I-4 hydrochloride anhydrous.

FIG. 6 depicts the DSC scan of compound I-4 hydrochloride anhydrous.

### DETAILED DESCRIPTION OF CERTAIN EMBODIMENTS

Compounds of this invention include those described generally above, and are further illustrated by the classes, subclasses, and species disclosed herein. In some embodiments, provided compounds are agonists of the α7 nicotinic acetylcholine receptor. For purposes of this invention, the chemical elements are identified in accordance with the Periodic Table of the Elements, CAS version, Handbook of Chemistry and Physics, 75^{th} Ed. Additionally, general principles of organic chemistry are described in Organic Chemistry, Thomas Sorrell, University Science Books, Sausalito: 1999, and March's Advanced Organic Chemistry, 5^{th} Ed., Ed.: Smith, M. B. and March, J., John Wiley & Sons, New York: 2001, the entire contents of which are hereby incorporated by reference.

It has been surprisingly found that certain compounds of the present disclosure have improved agonist activity against α7 nAChR, improved selectivity against the nicotinic nAChR α3 subtype, and an improved cytochrome P450 profile.

In some embodiments, provided compounds have improved agonist activity against α7 nAChR. In certain embodiments, provided compounds have improved selectivity against the nicotinic nAChR α3 subtype. In some embodiments, provided compounds have an improved cytochrome P450 profile. In some embodiments, provided compounds are more potent agonists against α7 nAChR than they are antagonists against α3 nAChR (i.e., they are more selective for the α7 subtype compared to the α3 subtype).

In certain embodiments, the present invention provides compounds of formula I:

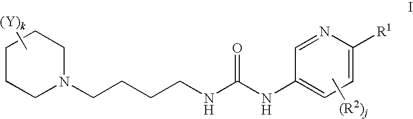

or a pharmaceutically acceptable salt thereof,
wherein,
j is 0 or 1;
k is 0 or 1;
R¹ is selected from the group consisting of phenyl, furanyl, thienyl, pyrazolyl, pyridyl, pyrimidyl, benzofuranyl, and benzodioxyl; wherein a carbon atom of R¹ is attached to the pyridyl group, and R¹ is optionally substituted with 1 to 3 groups independently selected from the group consisting of halogen, C₁₋₃ alkyl, and C₁₋₃ alkoxy;
R² is halogen or a linear or branched group selected from C₁₋₃ alkyl or C₁₋₃ alkoxy; and
Y is —OH or ═O; or
   Y forms an N-oxide moiety when linked directly to the piperidine nitrogen; with the proviso that Y is not ═O when its position relative to the piperidine nitrogen transforms the ring into a lactam ring.

In certain embodiments, R¹ is optionally substituted phenyl. In certain embodiments, R¹ is optionally substituted furanyl. In certain embodiments, R¹ is optionally substituted thienyl. In certain embodiments, R¹ is optionally substituted pyrazolyl. In certain embodiments, R¹ is optionally substituted pyridyl. In certain embodiments, R¹ is optionally substituted pyrimidyl. In certain embodiments, R¹ is optionally substituted benzofuranyl. In certain embodiments, R¹ is optionally substituted benzodioxyl.

In some embodiments, R¹ is substituted with 1 to 3 groups independently selected from the group consisting of halogen, C₁₋₃ alkyl, and C₁₋₃ alkoxy. In some embodiments, R¹ is substituted with 1 group selected from the group consisting of halogen, C₁₋₃ alkyl, and C₁₋₃ alkoxy. In some embodiments, R¹ is substituted with 2 groups independently selected from the group consisting of halogen, C₁₋₃ alkyl, and C₁₋₃ alkoxy. In some embodiments, R¹ is substituted with 3 groups independently selected from the group consisting of halogen, C₁₋₃ alkyl, and C₁₋₃ alkoxy.

In some embodiments, R¹ is substituted with a halogen group. In some embodiments, R¹ is substituted with a chloro group. In some embodiments, R¹ is substituted with a fluoro group. In some embodiments, R¹ is substituted with a bromo group. In some embodiments, R¹ is substituted with an iodo group.

In some embodiments, R¹ is substituted with a C₁₋₃ alkyl group. In some embodiments, R¹ is substituted with a methyl group.

In some embodiments, R¹ is substituted with a C₁₋₃ alkoxy group. In some embodiments, R¹ is substituted with a —OMe group. In some embodiments, R¹ is substituted with a —OEt group.

In certain embodiments, R² is halogen. In some embodiments, R² is fluoro. In some embodiments, R² is chloro. In some embodiments, R² is bromo. In some embodiments, R² is iodo. In some embodiments, R² is a linear or branched C₁₋₃ alkyl group. In some embodiments, R² is a linear or branched C₁-₃ alkoxy group.

In certain embodiments, Y is —OH. In some embodiments, Y is ═O. In certain embodiments, Y is directly linked to the piperidine nitrogen to form a compound for of formula II:

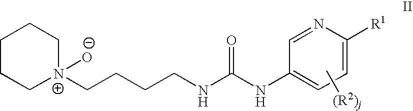

wherein each of j, R¹, and R² is as defined above for compounds of formula I.

In some embodiments, Y is other than ═O when its position relative to the piperidine nitrogen transforms the ring into a lactam ring.

In certain embodiments, j is 0. In other embodiments, j is 1.

In certain embodiments, k is 0. In other embodiments, k is 1.

Exemplary compounds of formula I are set forth in Table 1, below.

TABLE 1

I-1

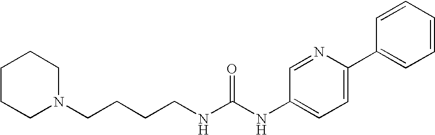

I-2

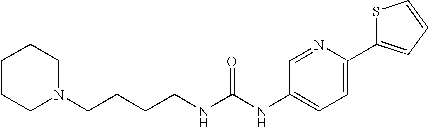

I-3

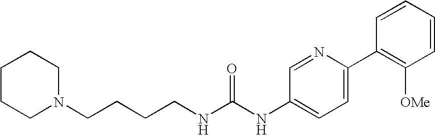

I-4

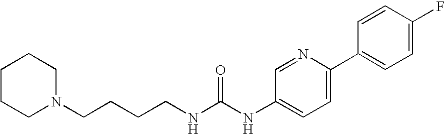

I-5

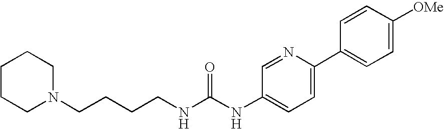

I-6

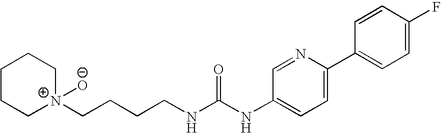

I-7

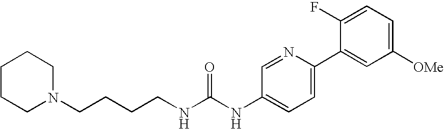

I-8

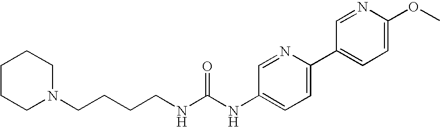

I-9

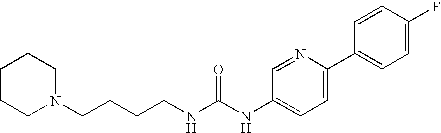

I-10

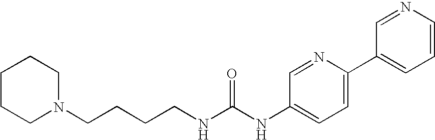

I-11

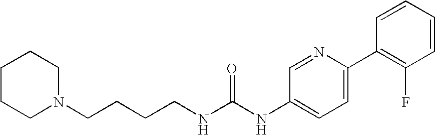

I-12

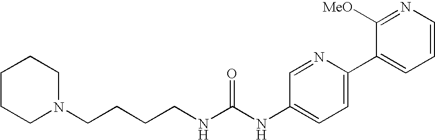

I-13

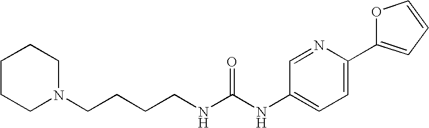

I-14

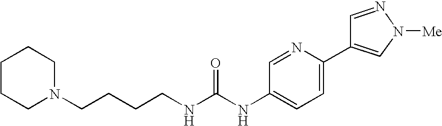

I-15

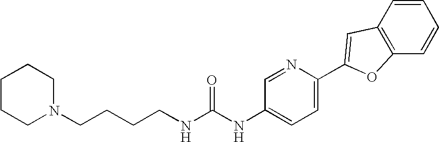

I-16

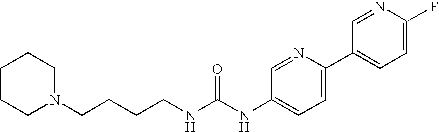

I-17

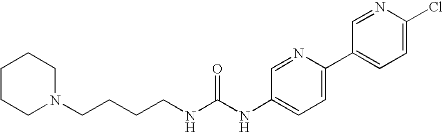

I-18

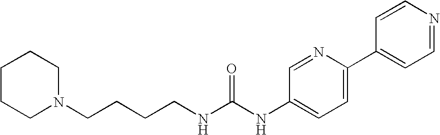

I-19

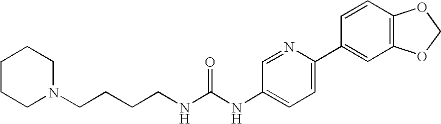

I-20

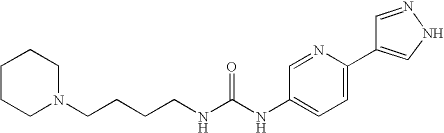

I-21

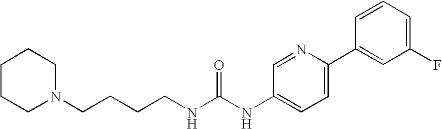

I-22

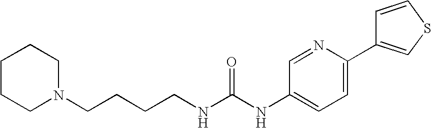

I-23

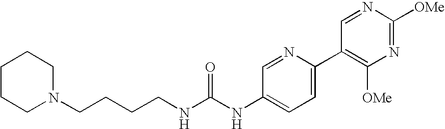

In some embodiments, compounds of the present invention have improved agonist activity against α7 nAChR. In some embodiments, compounds of the present invention have improved selectivity against the nicotinic nAChR α3 subtype. In some embodiments, compounds of the present invention have an improved cytochrome P450 profile. Biological evaluation of compounds of formula I is described quantitatively in examples set forth herein.

### Synthesis of Compounds

Compounds of the invention may be synthesized according to the schemes described below. The reagents and conditions described are intended to be exemplary and not limiting. As one of skill in the art would appreciate, various analogs may be prepared by modifying the synthetic reactions such as using different starting materials, different reagents, and different reaction conditions (e.g., temperature, solvent, concentration, etc.)

In one aspect, the present invention provides methods for the synthesis of compounds of formula I and intermediates thereto. In some embodiments, such methods are as shown in Scheme A, below:

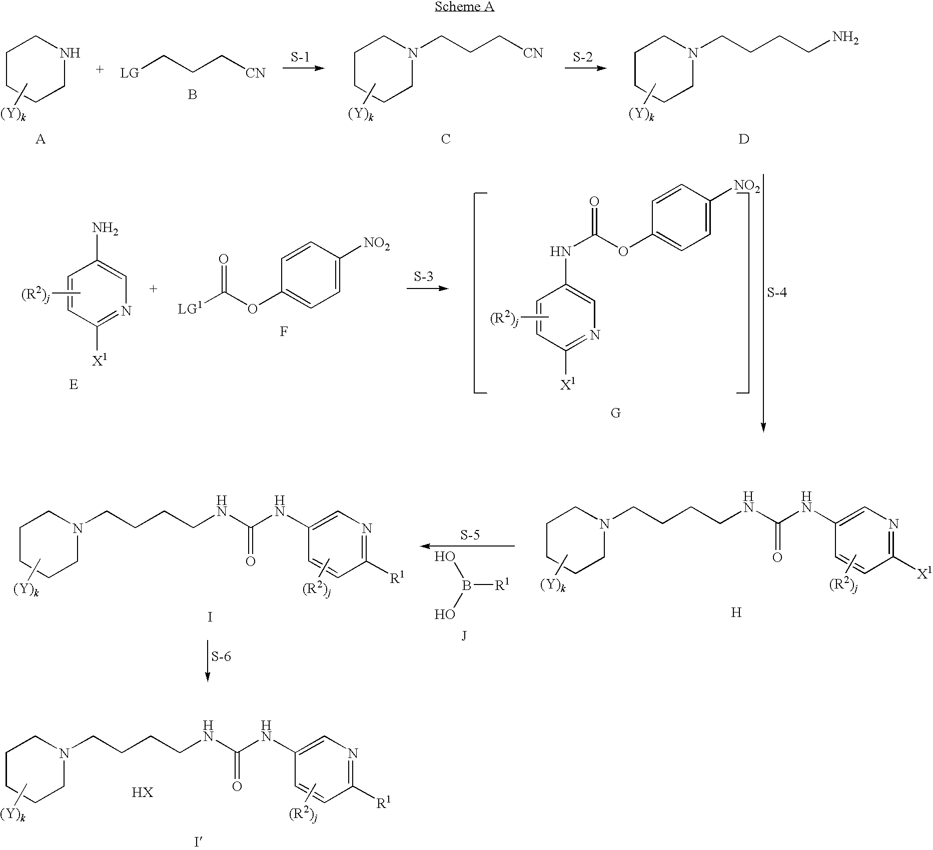

wherein each of j, k, Y, R¹ and R² is as defined above and described in classes and subclasses herein; and LG, LG¹, and X¹ are described below.

At step S-1, piperidine of formula A is reacted with nitrile of formula B under suitable conditions to form piperidine of formula C. The LG group of formula B is a suitable leaving group. One of ordinary skill in the art will appreciate that a variety of suitable leaving groups LG can be used to facilitate the reaction described in step S-1, and all such suitable leaving groups are contemplated by the present invention. A suitable leaving group is a chemical group that is readily displaced by a desired incoming chemical moiety. Suitable leaving groups are well known in the art, e.g., see, March, supra. Such leaving groups include, but are not limited to, halogen, alkoxy, sulphonyloxy, optionally substituted alkylsulphonyl, optionally substituted alkenylsulfonyl, optionally substituted arylsulfonyl, and diazonium moieties. Examples of some suitable leaving groups include chloro, iodo, bromo, fluoro, methanesulfonyl (mesyl), tosyl, triflate, nitro-phenylsulfonyl (nosyl), and bromo-phenylsulfonyl (brosyl).

Step S-1 may optionally employ a suitable base. Such suitable bases include inorganic bases and amine bases.

Solvents suitable for use in step S-1 include halogenated hydrocarbons (e.g., dichloromethane, chloroform, carbon tetrachloride, methyl chloroform, 1,2-dichloroethane, 1,1-dichloroethane), aromatic hydrocarbons (e.g., benzene, toluene, xylenes, ethylbenzene) or halogenated aromatic hydrocarbons (e.g., chlorobenzene, dichlorobenzenes). In certain embodiments, the solvent is toluene.

In some embodiments, step S-2 is carried out at temperatures of about 20-70° C. In some embodiments, the temperature is about 50-70° C. In some embodiments, the temperature is about 60° C.

In some embodiments, the present invention provides a method comprising the steps of:
(a) providing a piperidine of formula A:

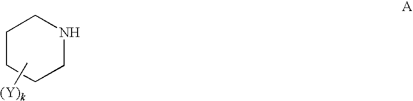

wherein
k is 0 or 1; and
Y is —OH or ═O; with the proviso that Y is not ═O when its position relative to the piperidine nitrogen transforms the ring into a lactam ring; and with the proviso that Y is not directly attached to the piperidine nitrogen;
and
(b) reacting the piperidine of formula A under suitable conditions with a nitrile of formula B:

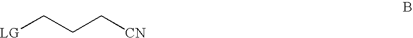

wherein LG is a suitable leaving group;
to form piperidine of formula C:

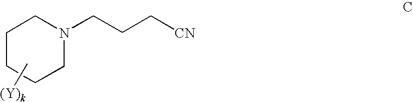

At step S-2, a piperidine of formula C is treated under suitable reducing conditions to form an amine of formula D. In certain embodiments, the reduction reaction is a hydrogenation reaction conducted in the presence of hydrogen gas and a metal catalyst. In certain embodiments, the metal catalyst is palladium on carbon or with ZnBr₂, Pt/C, Ru/C, Rh/C, PtO₂. In some embodiments, the palladium catalyst is palladium (II) hydroxide. In some embodiments, the hydrogenation reaction can be run in methanol, ethanol, ethyl acetate, or acetic acid, THF, isopropanol. In some embodiments, the hydrogenation is conducted in the presence of sulfuric acid, acetic acid, or both. In some embodiments, the hydrogenation is conducted in the presence of ammonium hydroxide. Suitable hydrogenation or reducing conditions are well known in the art and include those described by March (supra). Additional suitable reducing agents include, but are not limited to, H₂ (g) with palladium or platinum catalysts, cyclohexene with Pd/C (catalytic transfer hydrogenation), Zn/HCl, Li/NH₃, Raney Ni, trialkylsilyl hydride (e.g., Et₃SiH), sodium borohydride, or lithium aluminum hydride, or the like. In certain embodiments, the catalyst is Raney Ni.

In some embodiments, the hydrogenation reaction is run in methanol, ethanol, ethyl acetate, or acetic acid, THF, isopropanol. In certain embodiments, the solvent is methanol.

In certain embodiments, the hydrogenation reaction, described above and herein, is conducted at pressures at about 50 psi (H₂) or above. In some embodiments, the hydrogenation reaction is conducted at about 50 psi H₂. In some embodiments, the hydrogenation reaction is conducted at about 60 psi H₂. In some embodiments, the hydrogenation reaction is conducted at about 70 psi H₂.

In certain embodiments, the hydrogenations are conducted with heating of the reaction mixture. In some embodiments, the hydrogenations are conducted at temperatures between about 30° C. and about 50° C.

In certain embodiments, the present invention provides a method comprising the steps of:
(a) providing a piperidine of formula C:

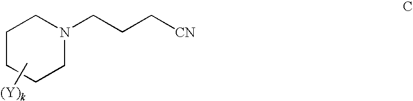

and
(b) reacting the piperidine of formula C under suitable hydrogenation conditions to form an amine of formula D:

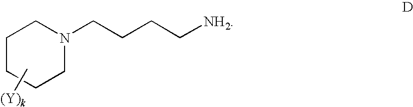

At step S-3, an aniline of formula E is reacted under suitable conditions with a compound of formula F to form a carbamate of formula G. For compounds of formula F, LG¹ is a suitable leaving group as defined above for LG. Step S-3 may employ a suitable base. Such suitable bases are known in the art and will vary upon the choice of LG¹. In some embodiments, the base is an amine base.

Step S-3 may employ a suitable solvent. Solvents suitable for use in step S-3 include polar aprotic solvents (i.e., THF, dioxane, acetonitrile, and combinations thereof), halogenated hydrocarbons (e.g., dichloromethane, chloroform, carbon tetrachloride, methyl chloroform, 1,2-dichloroethane, 1,1-dichloroethane), aromatic hydrocarbons (e.g., benzene, toluene, xylenes, ethylbenzene) or halogenated aromatic hydrocarbons (e.g., chlorobenzene, dichlorobenzenes). In some embodiments, the solvent is acetonitrile.

In some embodiments, step S-3 is carried out at temperatures of about 20-60° C. In certain embodiments, the temperature is about 35° C.

In certain embodiments, the present invention provides a method comprising the steps of:
(a) providing an aniline of formula E:

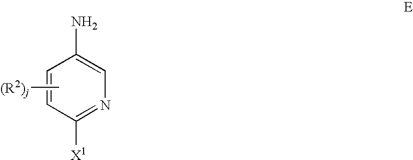

wherein
j is 0 or 1;
R² is halogen or a linear or branched group selected from C₁-₃ alkyl or C₁-₃ alkoxy; and
X¹ is selected from chloro, iodo, bromo, fluoro, methanesulfonyl (mesyl), tosyl, or triflate;
and
(b) reacting the aniline of formula E under suitable conditions with a compound of formula F:

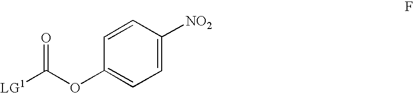

wherein LG¹ is a suitable leaving group;
to form a carbamate of formula G:

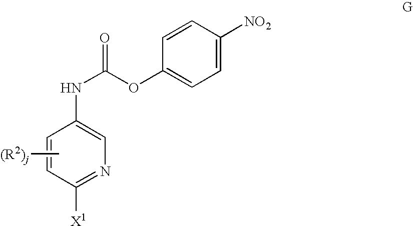

or a salt thereof.

At step S-4, the carbamate formed in step S-3 is reacted with an amine of formula D to form a urea of formula H. Step S-4 may be performed without isolation of the product of step S-3. In some embodiments, an amine of formula D is added to a carbamate of formula G without isolation of the intermediate carbamate of formula G. In some embodiments, a carbamate of formula G is generated in situ and then added to an amine of formula D. In some embodiments, a carbamate of formula G is a salt. In some embodiments, a carbamate of formula G is a hydrochloric salt.

Step S-4 may employ a suitable solvent. Solvents suitable for use in step S-4 include polar aprotic solvents (i.e., THF, DMF, dioxane, acetonitrile, and combinations thereof), halogenated hydrocarbons (e.g., dichloromethane, chloroform, carbon tetrachloride, methyl chloroform, 1,2-dichloroethane, 1,1-dichloroethane), aromatic hydrocarbons (e.g., benzene, toluene, xylenes, ethylbenzene) or halogenated aromatic hydrocarbons (e.g., chlorobenzene, dichlorobenzenes). In some embodiments, the solvent is acetonitrile.

In some embodiments, step S-4 is carried out at temperatures of about 20-60° C. In certain embodiments, the temperature is about 35° C.

In certain embodiments, the present invention provides a method comprising the steps of:
(a) providing a carbamate of formula G:

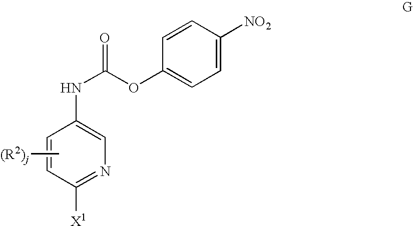

and
(b) reacting the carbamate of formula G under suitable conditions with an amine of formula D:

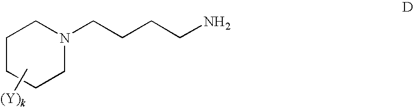

to form a urea of formula H:

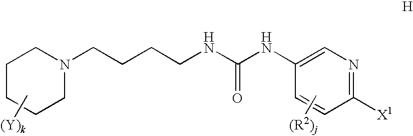

or a salt thereof.

At step S-5, a urea of formula H is reacted with a boronic acid of formula J to form a urea of formula I. Methods of carrying out Suzuki couplings are well known in the art and include those described by March (supra). Suitable conditions for the Suzuki reaction employ a palladium catalyst. In certain embodiments the catalyst is Pd(OAc)₂/PPh₃. In certain embodiments the catalyst is Pd/C/PPh₃. In certain embodiments the catalyst is Pd-118 (dtbpfPdCl₂). In some embodiments, the catalyst is PdCl₂[(PPh₃)]₂. In some embodiments, the catalyst is Pd[(PPh₃)]₄.

In some embodiments, the boronic acid is a boronic ester.

In some embodiments, the amount of catalyst used is about 0.005 mol % to about 5 mol %. In some embodiments, the amount of catalyst used is about 0.01 mol % to about 1 mol %. In some embodiments, the amount of catalyst used is about 0.01 mol % to about 0.1 mol %. In some embodiments, the amount of catalyst used is about 0.01 mol % to about 0.05 mol %. In some embodiments, the amount of catalyst used is about 0.03 mol %.

Step S-5 typically employs a base. In some embodiments, the base is K₂CO₃. In some embodiments, the base is Cs₂CO₃. In some embodiments, the base is Na₂CO₃.

Step S-5 typically employs a suitable solvent. Examples of solvents suitable for use at step S-5 include polar solvents such as alkyl alcohols, for example C₁ to C₄ alcohols (e.g. ethanol, methanol, 2-propanol), aromatic hydrocarbons, dioxane, ethyl acetate, acetonitrile, THF (tetrahydrofuran) or combinations thereof. In certain embodiments, the solvent is ethanol. In certain embodiments, the solvent is toluene. In certain embodiments, the solvent is DME. In certain embodiments, the solvent is DMF. In certain embodiments, the solvent is THF. In certain embodiments, the solvent is M-THF. In certain embodiments, the solvent is MeCN.

In certain embodiments, the present invention provides a method comprising the steps of:
(a) providing a urea of formula H:

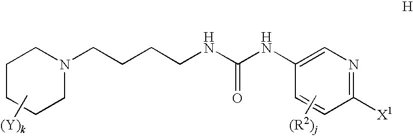

and
(b) reacting the urea of formula H under suitable conditions with a boronic acid of formula J:

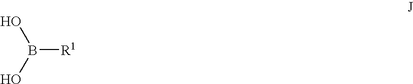

wherein R¹ is selected from the group consisting of phenyl, furanyl, thienyl, pyrazolyl, pyridyl, pyrimidyl, benzofuranyl, and benzodioxyl; wherein a carbon atom of R¹ is attached to the pyridyl group, and R¹ is optionally substituted with 1 to 3 groups independently selected from the group consisting of halogen, C₁₋₃ alkyl, and C₁₋₃ alkoxy;
to form a compound of formula I:

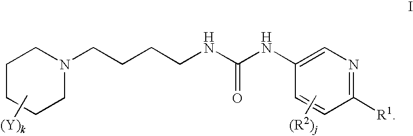

At step S-6, a compound of formula I is reacted under suitable conditions with a suitable acid to form a salt of formula I′. In certain embodiments, the acid is selected such that the resulting salt of formula I′ is a pharmaceutically acceptable salt as described herein (infra). In certain embodiments, the acid is hydrochloric. In certain embodiments, the salt is a hydrochloride salt. In some embodiments, the hydrochloride salt is amorphous. In some embodiments, the salt is hydrochloride salt is crystalline. In some embodiments, the hydrochloride salt is anhydrous. In some embodiments, the hydrochloride salt is a hydrate. In some embodiments, the hydrochloride salt is a monohydrate.

One of ordinary skill in the art will appreciate that suitable solvents for carrying out a crystallization of step S-6 include, for example, methanol, ethanol, isopropanol, dichloromethane, acetonitrile, ethyl acetate, hexanes, heptane, tetrahydrofuran, cyclohexane, benzene, toluene, xylenes, diethyl ether, tert-butyl methyl ether, water, or a mixture thereof.

In some embodiments, the crystallization is achieved from a protic solvent. In some embodiments, the protic solvent is an alcohol. It will be appreciated that the crystallization may be achieved using a single protic solvent or a combination of one or more protic solvents. Such solvents and solvent mixtures are well known to one of ordinary skill in the art and include one or more straight or branched alkyl alcohols. In certain embodiments, the crystallization is achieved from ethyl alcohol.

In certain embodiments, the present invention provides a method comprising the steps of:
(a) providing a compound of formula I:

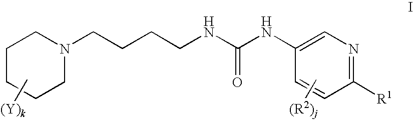

and
(b) treating the compound of formula I under suitable conditions to provide a compound of formula I′:

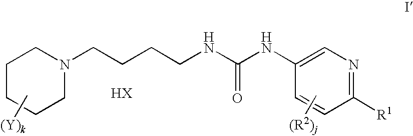

wherein X is a suitable counterion.

It will be appreciated that for compounds described in Scheme A, k may be 0 or 1. In certain embodiments, k is 1. One of ordinary skill in the art will recognize that it may be necessary to protect an —OH group on the piperidine ring in order to carry out the described synthesis. Such hydroxyl protecting groups are known in the art and are described in detail in Protecting Groups in Organic Synthesis, T. W. Greene and P. G. M. Wuts, 3^{rd} edition, John Wiley & Sons, 1999, the entirety of which is incorporated herein by reference.

It will be appreciated that, for any intermediate of Scheme A, the piperidine group can be subjected to suitable conditions to form an N-oxide. Methods of forming N-oxides are known in the art and include those described herein. In some embodiments, an N-oxide is made from a compound of formula H. In some embodiments, an N-oxide is made from a compound of formula I. In some embodiments, an N-oxide is made from a compound of formula I′.

In certain embodiments, each of the aforementioned synthetic steps may be performed sequentially with isolation of each intermediate performed after each step. Alternatively, each of steps S-1, S-2, S-3, S-4, S-5, and S-6, as depicted in Scheme A above, may be performed in a manner whereby no isolation of one or more intermediates A, B, C, D, E, F, G, H, or I is performed.

In certain embodiments, all the steps of the aforementioned synthesis may be performed to prepare the desired final product. In other embodiments, two, three, four, five, or more sequential steps may be performed to prepare an intermediate or the desired final product.

### Provided Forms

In certain embodiments, the present invention provides compound I-4 or a pharmaceutically acceptable salt thereof. Compound I-4 has strong and specific activity as a modulator of α7 nicotinic acetylcholine receptors.

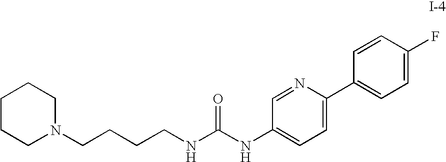

In some embodiments, the present invention provides salt forms of compound I-4. For example, as described herein, the present invention provides acetic, citric, D-glucuronic, fumaric, hydrochloric, oxalic, maleic, phosphoric, salicylic, succinic, sulfuric, and tartaric acid forms. The present invention particularly provides solid forms of certain salts of compound I-4. For example, the present invention provides solid forms of the hydrochloride salt of compound I-4, referred to herein as “compound I-4 hydrochloride.”

The present invention also demonstrates that discrete crystalline forms of the hydrochloride salt of compound I-4 can be achieved. Among others, the present invention specifically exemplifies a monohydrate form of compound I-4 hydrochloride, referred to herein as “compound I-4 hydrochloride monohydrate.” In certain embodiments, the present invention specifically exemplifies an anhydrous form of compound I-4 hydrochloride, referred to herein as “compound I-4 hydrochloride anhydrous.”

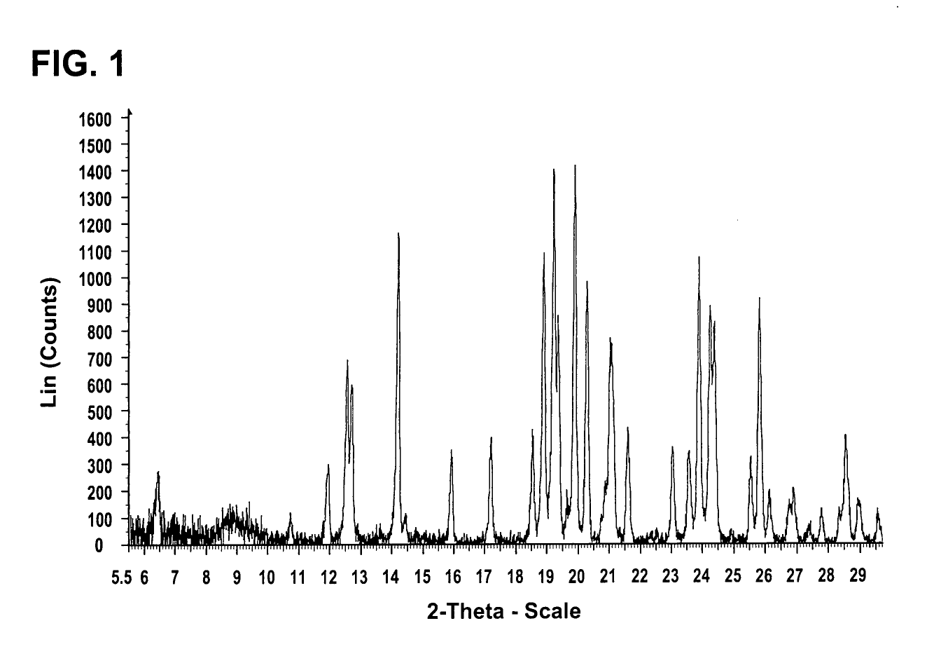

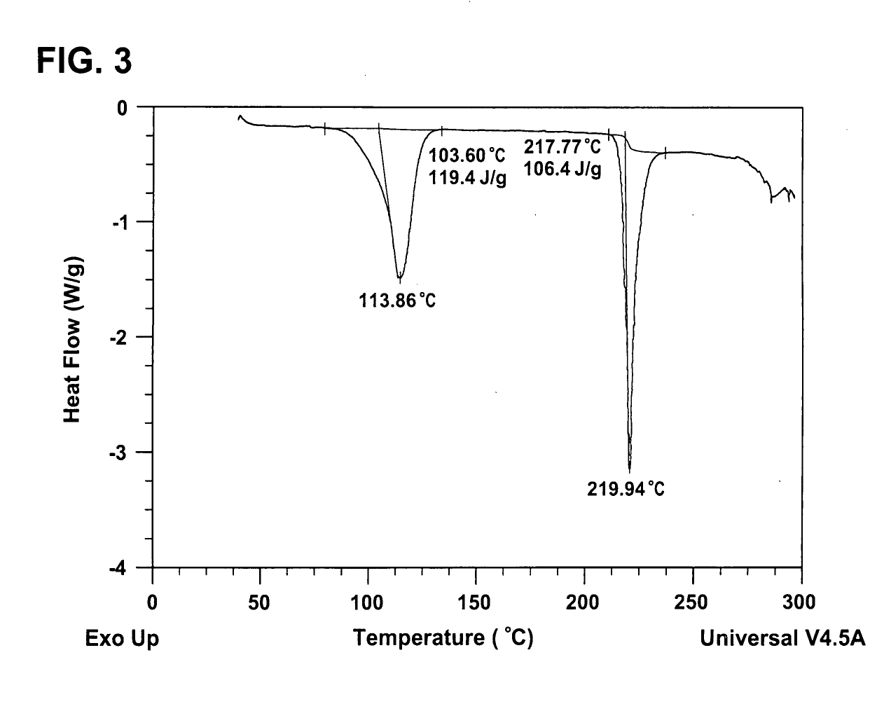

As described herein, compound I-4 hydrochloride monohydrate may be characterized by, for example, two endotherms, one in the range of 70-120° C., and a second one at an onset temperature of around 218° C. as depicted in the DSC scan shown in FIG. 3, and/or by an X-ray diffraction (“XRD”) pattern as shown for example in FIG. 1. In certain embodiments, compound I-4 hydrochloride monohydrate is characterized in that the form has at least one peak in its XRD pattern selected from about 12.5, 14.2, 19.2, 23.8, or 25.8 degrees 2-theta. In some embodiments, compound I-4 hydrochloride monohydrate is characterized in that the form has at least two peaks in its XRD pattern selected from about 12.5, 14.2, 19.2, 23.8, or 25.8 degrees 2-theta. In other embodiments, compound I-4 hydrochloride monohydrate is characterized in that is has substantially all of the peaks in its XRD pattern listed in Table 2, below.

TABLE 2
XRD Peaks for Compound I-4 Hydrochloride Monohydrate

| Angle | d value | Intensity % |
|---|---|---|
| 2-Theta ° | Angstrom | % |
| 6.4 | 13.9 | 19.1 |
| 10.7 | 8.3 | 8.0 |
| 11.9 | 7.4 | 21.3 |
| 12.5 | 7.1 | 49.1 |
| 12.7 | 7.0 | 42.3 |
| 14.2 | 6.3 | 81.8 |
| 14.4 | 6.1 | 8.6 |
| 15.9 | 5.6 | 24.1 |
| 17.2 | 5.2 | 28.7 |
| 18.5 | 4.8 | 30.2 |
| 18.9 | 4.7 | 76.5 |
| 19.2 | 4.6 | 99.4 |
| 19.3 | 4.6 | 60.2 |
| 19.6 | 4.5 | 14.8 |
| 19.9 | 4.5 | 100.0 |
| 20.2 | 4.4 | 69.8 |
| 21.0 | 4.2 | 54.3 |
| 21.5 | 4.1 | 30.9 |
| 23.0 | 3.9 | 25.6 |
| 23.5 | 3.8 | 24.7 |
| 23.8 | 3.7 | 75.6 |
| 24.2 | 3.7 | 62.7 |
| 24.3 | 3.7 | 58.6 |
| 25.5 | 3.5 | 23.1 |
| 25.8 | 3.5 | 65.1 |
| 26.1 | 3.4 | 14.5 |
| 26.7 | 3.3 | 12.0 |
| 26.9 | 3.3 | 15.4 |
| 27.8 | 3.2 | 9.6 |
| 28.6 | 3.1 | 29.0 |
| 29.0 | 3.1 | 12.7 |
| 28.3 | 3.1 | 9.9 |
| 29.6 | 3.0 | 9.6 |

In certain embodiments of the invention, compound I-4 hydrochloride monohydrate is characterized by representative peaks in XRD, which peaks are determined by comparison of XRD pattern results for standard preparations of compound I-4 hydrochloride monohydrate and compound I-4 hydrochloride anhydrous.

According to one aspect, compound I-4 hydrochloride monohydrate has an XRD pattern substantially similar to that depicted in FIG. 1. As used herein, the phrase “substantially all of the peaks” means that the compound exhibits, in its XRD, at least about 80% of the peaks listed. In other embodiments, the phrase “substantially all of the peaks” means that the compound exhibits, in its XRD, at least about 85, 90, 95, 97, 98, or 99% of the peaks listed. In other embodiments, compound I-4 hydrochloride monohydrate is characterized in that it has a DSC pattern substantially similar to that depicted in FIG. 3.

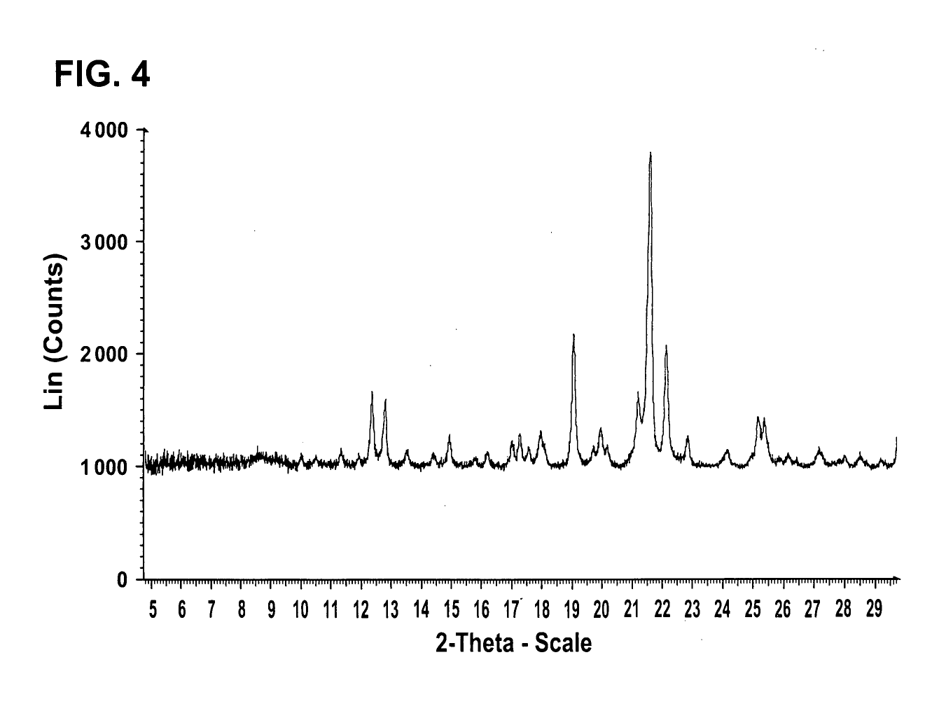

As described herein, compound I-4 hydrochloride anhydrous may be characterized by, for example, a melting point at an onset temperature of around 214° C. and/or by an X-ray diffraction pattern as shown for example in FIG. 4. In certain embodiments, a compound I-4 anhydrous is characterized in that the form has at least one peak in its XRD pattern selected from about 10.0, 11.3, 12.3, 14.9, 17.0, 17.5, 17.9, 19.0, 21.5, or 22.8 degrees 2-theta. In certain embodiments, a compound I-4 anhydrous is characterized in that the form has at least two peaks in its XRD pattern selected from about 10.0, 11.3, 12.3, 14.9, 17.0, 17.5, 17.9, 19.0, 21.5, or 22.8 degrees 2-theta. In certain embodiments, a compound I-4 hydrochloride anhydrous may be characterized by substantially all of the XRD peaks at 2 degrees theta as recited in Table 3, below.

TABLE 3
XRD Peaks for Compound I-4 Hydrochloride Anhydrous

| Angle | d value | Intensity % |
|---|---|---|
| 2-Theta ° | Angstrom | % |
| 29.8 | 3.0 | 39.0 |
| 28.5 | 3.1 | 30.6 |
| 27.2 | 3.3 | 30.0 |
| 26.1 | 3.4 | 29.3 |
| 25.4 | 3.5 | 37.7 |
| 25.2 | 3.5 | 37.8 |
| 24.1 | 3.7 | 30.0 |
| 22.8 | 3.9 | 33.4 |
| 22.1 | 4.0 | 54.5 |
| 21.5 | 4.1 | 100.0 |
| 21.2 | 4.2 | 43.8 |
| 20.1 | 4.4 | 31.3 |
| 19.9 | 4.5 | 35.2 |
| 19.7 | 4.5 | 31.0 |
| 19.0 | 4.7 | 57.3 |
| 17.9 | 4.9 | 34.6 |
| 17.5 | 5.1 | 30.5 |
| 17.2 | 5.1 | 33.9 |
| 17.0 | 5.2 | 31.9 |
| 16.2 | 5.5 | 29.7 |
| 15.8 | 5.6 | 28.0 |
| 14.9 | 5.9 | 33.6 |
| 14.4 | 6.2 | 29.3 |
| 13.5 | 6.6 | 29.9 |
| 12.8 | 6.9 | 41.2 |
| 12.3 | 7.2 | 43.8 |
| 11.3 | 7.8 | 30.3 |
| 10.0 | 8.9 | 29.2 |

In certain embodiments of the invention, compound I-4 hydrochloride anhydrous is characterized by representative peaks in XRD, which peaks are determined by comparison of XRD pattern results for standard preparations of compound I-4 hydrochloride anhydrous and compound I-4 hydrochloride monohydrate.

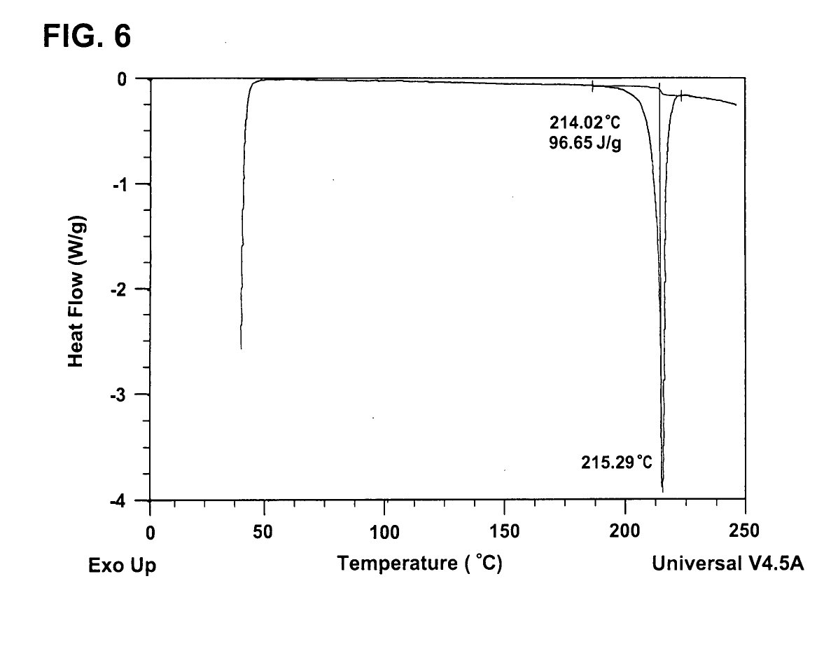

According to one aspect, compound I-4 hydrochloride anhydrous has an XRD pattern substantially similar to that depicted in FIG. 4. In other embodiments, compound I-4 hydrochloride anhydrous is characterized in that it has a DSC pattern substantially similar to that depicted in FIG. 6.

According to another embodiment, the present invention provides compound I-4 hydrochloride as an amorphous solid. Amorphous solids are well known to one of ordinary skill in the art and are typically prepared by such methods as lyophilization, melting, and precipitation from supercritical fluid, among others.

In certain embodiments, the present invention provides amorphous compound I-4 hydrochloride substantially free of crystalline compound I-4 hydrochloride. As used herein, the term “substantially free of crystalline compound I-4 hydrochloride” means that the compound contains no significant amount of crystalline compound I-4 hydrochloride. Crystalline compound I-4 hydrochloride includes neat crystal forms, solvates and hydrates as described herein or other crystalline forms of compound I-4 hydrochloride that may result from the preparation of, and/or isolation of, amorphous compound I-4 hydrochloride. In certain embodiments of the present invention, at least about 95% by weight of compound I-4 hydrochloride present is amorphous compound I-4 hydrochloride. In still other embodiments of the invention, at least about 99% by weight of compound I-4 hydrochloride present is amorphous compound I-4 hydrochloride.

In other embodiments, the present invention provides a composition comprising amorphous compound I-4 hydrochloride and at least one crystalline form of compound I-4 hydrochloride. Such crystalline forms of compound I-4 hydrochloride include monohydrate and anhydrous forms as described herein. In certain embodiments, the present invention provides a composition comprising amorphous compound I-4 hydrochloride and at least one crystalline form of compound I-4 hydrochloride as described herein.

Those of ordinary skill in the art will appreciate that X-ray diffraction patterns are often used to characterize individual crystal forms of a particular compound, and/or to detect the presence of the particular form in a complex composition. Those of ordinary skill in the art will further appreciate that precise identity of all peaks is not required to reveal a match of crystal form. Rather, presence or absence of particular characteristic peaks, and/or patterns of peaks and intensities, are typically both necessary and sufficient to characterize and/or identify a particular form.

The present invention provides new forms of a compound of formula I. In some embodiments, the present invention provides solid forms of a compound of formula I. Indeed, the present invention encompasses the recognition that significant challenges can be encountered in preparing solid forms of a compound of formula I. For example, the free base form and many salt forms of the compound do not readily adopt a solid state, but rather are typically liquid or semi-solid. Moreover, their behaviors may not be reproducible. The present invention encompasses the recognition that there is a need for new forms of a compound of formula I, and also that there is a particular need for solid forms.

Pharmacological activity of a representative group of compounds of formula I was demonstrated in an in vitro assay utilising cells stably transfected with the alpha 7 nicotinic acetylcholine receptor and cells expressing the alpha 1 and alpha 3 nicotinic acetylcholine receptors and 5HT3 receptor as controls for selectivity.

Compounds of formula I may be provided according to the present invention in any of a variety of useful forms, for example as pharmaceutically acceptable salts, as particular crystal forms, etc. In some embodiments, prodrugs of one or more compounds of Formula (I) are provided. Various forms of prodrugs are known in the art, for example as discussed in Bundgaard (ed.), Design of Prodrugs, Elsevier (1985); Widder et al. (ed.), Methods in Enzymology, vol. 4, Academic Press (1985); Kgrogsgaard-Larsen et al. (ed.); “Design and Application of Prodrugs”, Textbook of Drug Design and Development, Chapter 5, 113-191 (1991); Bundgaard et al., Journal of Drug Delivery Reviews, 8:1-38 (1992); Bundgaard et al., J. Pharmaceutical Sciences, 77:285 et seq. (1988); and Higuchi and Stella (eds.), Prodrugs as Novel Drug Delivery Systems, American Chemical Society (1975).

### DEFINITIONS

The term “aliphatic” or “aliphatic group,” as used herein, means a straight-chain (i.e., unbranched) or branched, hydrocarbon chain that is completely saturated or that contains one or more units of unsaturation, or a monocyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. In certain embodiments, aliphatic groups contain 1-6 aliphatic carbon atoms, and in yet other embodiments, aliphatic groups contain 1-3 aliphatic carbon atoms. In some embodiments, “cycloaliphatic” (or “carbocycle”) refers to a monocyclic C₃₋₆ hydrocarbon that is completely saturated or that contains one or more units of unsaturation, but which is not aromatic, that has a single point of attachment to the rest of the molecule. Such cycloaliphatic groups include cycloalkyl and cycloalkenyl groups. Suitable aliphatic groups include, but are not limited to, linear or branched alkyl, alkenyl, alkynyl groups and hybrids thereof such as (cycloalkyl)alkyl, (cycloalkenyl)alkyl or (cycloalkyl)alkenyl.

The term “lower alkyl,” as used herein, refers to a hydrocarbon chain having up to 6 carbon atoms. In some embodiments, the lower alkyl chain has 1 to 3 carbon atoms. In some embodiments, the lower alkyl chain has 1 to 2 carbon atoms. The term “alkyl” includes, but is not limited to, straight and branched chains such as methyl, ethyl, n-propyl, isopropyl, n-butyl, isobutyl, sec-butyl, or t-butyl.

The term “alkoxy,” as used herein, refers to the group —OR*, wherein R* is a lower alkyl group.

The terms “halogen” or “halo,” as used herein, refer to chlorine, bromine, fluorine or iodine.

The term “hydrate”, as used herein, has its art-understood meaning, referring to a crystal form adopted by a particular compound in which either a stoichiometric or non-stoichiometric amount of water is incorporated into the crystal lattice.

The phrase “in combination”, as used herein, refers to agents that are simultaneously administered to a subject. It will be appreciated that two or more agents are considered to be administered “in combination” whenever a subject is simultaneously exposed to both (or more) of the agents. Each of the two or more agents may be administered according to a different schedule; it is not required that individual doses of different agents be administered at the same time, or in the same composition. Rather, so long as both (or more) agents remain in the subject's body, they are considered to be administered “in combination”.

As used herein, the term “polymorph” has its art-understood meaning, referring to one of a variety of different crystal structures that can be adopted by a particular compound.

As used herein, the term “solvate” has its art-understood meaning, referring to a crystal form adopted by a particular compound in which either a stoichiometric or non-stoichiometric amount of solvent is incorporated into the crystal lattice.

The term “substantially free of”, as used herein, means containing no more than an insignificant amount. In some embodiments, a composition or preparation is “substantially free of” a recited element if it contains less than 5%, 4%, 3%, 2%, or 1%, by weight of the element. In some embodiments, the composition or preparation contains less than 0.9%, 0.8%, 0.7%, 0.6%, 0.5%, 0.4%, 0.3%, 0.2%, 0.1% or less ofthe recited element. In some embodiments, the composition or preparation contains an undetectable amount of the recited element.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable salts” or “pharmaceutically acceptable salt” includes acid addition salts, that is salts derived from treating compounds of formula I with an organic or inorganic acid such as, for example, hydrochloric, phosphoric, nitric, sulfuric, glycolic, pyruvic, salicylic, or similarly known acceptable acids. For example, S. M. Berge et al., describe pharmaceutically acceptable salts in detail in J. Pharmaceutical Sciences, 1977, 66, 1-19). Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, cinnamate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, mandelate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Unless otherwise stated, structures depicted herein are also meant to include all isomeric (e.g., enantiomeric, diastereomeric, and geometric (or conformational)) forms of the structure; for example, the R and S configurations for each stereocenter, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

If, for instance, a particular enantiomer of a compound of the present invention is desired, it may be prepared by asymmetric synthesis, chiral chromatography, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

### Uses

Agents that bind to nicotinic acetylcholine receptors have been indicated as useful in the treatment and/or prophylaxis of various diseases and conditions, particularly psychotic diseases, neurodegenerative diseases involving a dysfunction of the cholinergic system, and conditions of memory and/or cognition impairment, including, for example, schizophrenia, anxiety, mania, depression, manic depression, Tourette's syndrome, Parkinson's disease, Huntington's disease, cognitive disorders (such as Alzheimer's disease, Lewy Body Dementia, Amyotrophic Lateral Sclerosis, memory impairment, memory loss, cognition deficit, attention deficit, Attention Deficit Hyperactivity Disorder, ), and other uses such as treatment of nicotine addiction, inducing smoking cessation, treating pain (i.e., analgesic use), providing neuroprotection, and treating jetlag. See, e.g., WO 97/30998; WO 99/03850; WO 00/42044; WO 01/36417; Holladay et al., J. Med. Chem., 40:26, 4169-94 (1997); Schmitt et al., Annual Reports Med. Chem., Chapter 5, 41-51 (2000); Stevens et al., Psychopharmatology, (1998) 136: 320-27; and Shytle et al., Molecular Psychiatry, (2002), 7, pp. 525-535.

Thus, in accordance with the invention, there is provided a method of treating a patient, especially a human, suffering from any of psychotic diseases, neurodegenerative diseases involving a dysfunction of the cholinergic system, and/or conditions of memory and/or cognition impairment, including, for example, schizophrenia, anxiety, mania, depression, manic depression, Tourette's syndrome, Parkinson's disease, Huntington's disease, and/or cognitive disorders (such as Alzheimer's disease, Lewy Body Dementia, Amyotrophic Lateral Sclerosis, memory impairment, memory loss, cognition deficit, attention deficit, Attention Deficit Hyperactivity Disorder) comprising administering to the patient an effective amount of a compound according to Formula I.

Neurodegenerative disorders whose treatment is included within the methods of the present invention include, but are not limited to, treatment and/or prophylaxis of Alzheimer's diseases, Pick's disease (Friedland, Dementia, (1993) 192-203; Procter, Dement Geriatr Cogn Disord. (1999) 80-4; Sparks, Arch Neurol. (1991) 796-9; Mizukami, Acta Neuropathol. (1989) 52-6; Hansen, Am J Pathol. (1988) 507-18), diffuse Lewy Body disease, progressive supranuclear palsy (Steel-Richardson syndrome, see Whitehouse, J Neural Transm Suppl. (1987) 24:175-82; Whitehouse, Arch Neurol. (1988) 45(7):722-4; Whitehouse, Alzheimer Dis Assoc Disord. 1995;9 Suppl 2:3-5; Warren, Brain. 2005 February;128(Pt 2):239-49), multisystem degeneration (Shy-Drager syndrome), motor neuron diseases including amyotrophic lateral sclerosis (Nakamizo, Biochem Biophys Res Commun. (2005) 330(4), 1285-9; Messi, FEBS Lett. (1997) 411(1):32-8; Mohammadi, Muscle Nerve. (2002) October;26(4):539-45; Hanagasi, Brain Res Cogn Brain Res. (2002) 14(2):234-44; Crochemore, Neurochem Int. (2005) 46(5):357-68), degenerative ataxias, cortical basal degeneration, ALS-Parkinson's-Dementia complex of Guam, subacute sclerosing panencephalitis, Huntington's disease (Kanazawa, J Neurol Sci. (1985) 151-65; Manyam, J Neurol. (1990) 281-4; Lange, JNeurol. (1992) 103-4; Vetter, J Neurochem. (2003) 1054-63; De Tommaso, Mov Disord. (2004) 1516-8; Smith, Hum Mol Genet. (2006) 3119-31; Cubo, Neurology. (2006) 1268-71), Parkinson's disease, synucleinopathies, primary progressive aphasia, striatonigral degeneration, Machado-Joseph disease/spinocerebellar ataxia type 3, olivopontocerebellar degenerations, Gilles De La Tourette's disease, bulbar, pseudobulbar palsy, spinal muscular atrophy, spinobulbar muscular atrophy (Kennedy's disease), primary lateral sclerosis, familial spastic paraplegia, Werdnig-Hoffmann disease, Kugelberg-Welander disease, Tay-Sach's disease, Sandhoff disease, familial spastic disease, Wohlfart-Kugelberg-Welander disease, spastic paraparesis, progressive multifocal leukoencephalopathy, prion diseases (such as Creutzfeldt-Jakob, Gerstmann-Straussler-Scheinker disease, Kuru and fatal familial insomnia), and neurodegenerative disorders resulting from cerebral ischemia or infarction including embolic occlusion and thrombotic occlusion as well as intracranial hemorrhage of any type (including, but not limited to, epidural, subdural, subarachnoid and intracerebral), and intracranial and intravertebral lesions (including, but not limited to, contusion, penetration, shear, compression and laceration).

In addition, α7nACh receptor agonists, such as the compounds of the present invention can be used to treat age-related dementia and other dementias and conditions with memory loss including age-related memory loss, senility, vascular dementia, diffuse white matter disease (Binswanger's disease), dementia of endocrine or metabolic origin, dementia of head trauma and diffuse brain damage, dementia pugilistica, alcoholism related dementia (Korsakoff Syndrome) and frontal lobe dementia. See, e.g., WO 99/62505., Tomimoto Dement Geriatr Cogn Disord. (2005), 282-8; Tohgi—J Neural Transm. (1996), 1211-20; Casamenti, Neuroscience (1993) 465-71, Kopelman, Br J Psychiatry (1995) 154-73; Cochrane, Alcohol Alcohol. (2005) 151-4).

Amyloid precursor protein (APP) and Aβ peptides derived therefrom, e.g., Aβ1-42 and other fragments, are known to be involved in the pathology of Alzheimer's disease. The Aβ1-42 peptides are not only implicated in neurotoxicity but also are known to inhibit cholinergic transmitter function. Further, it has been determined that Aβ peptides bind to α7nACh receptors. The inflammatory reflex is an autonomic nervous system response to an inflammatory signal. Upon sensing an inflammatory stimulus, the autonomic nervous system responds through the vagus nerve by releasing acetylcholine and activating nicotinic α7 receptors on macrophages. These macrophages in turn release cytokines. Dysfunctions in this pathway have been linked to human inflammatory diseases including rheumatoid arthritis, diabetes and sepsis. Macrophages express the nicotinic α7 receptor and it is likely this receptor that mediates the cholinergic anti-inflammatory response. See for example Czura, C J et al., J. Intern. Med., (2005) 257(2), 156-66; Wang, H. et al Nature (2003) 421: 384-388; de Jonge British Journal of Pharmacology (2007) 151, 915-929. The mammalian sperm acrosome reaction is an exocytosis process important in fertilization of the ovum by sperm. Activation of an α7 nAChR on the sperm cell has been shown to be essential for the acrosome reaction (Son, J.-H. and Meizel, S. Biol. Reproduct. 68: 1348-1353, 2003). In addition, nicotinic receptors have been implicated as playing a role in the body's response to alcohol ingestion. α7nACh receptor agonists such as compounds provided herein, therefore, are also useful in the treatment of these disorders, diseases, and conditions.

A number of recent observations point to a potential neuroprotective effect of nicotine in a variety of neurodegeneration models in animals and in cultured cells, involving excitotoxic insults (Prendergast, M. A., et al. Med. Sci. Monit. (2001), 7, 1153-1160; Garrido, R., et al. (2001), J. Neurochem. 76, 1395-1403; Semba, J., et al. (1996) Brain Res. 735, 335-338; Shimohama, S., et al.(1996), Ann. N.Y. Acad. Sci. 777, 356-361; Akaike, A., et al. (1994) Brain Res. 644, 181-187), trophic deprivation (Yamashita, H., Nakamura, S. (1996) Neurosci. Lett. 213, 145-147), ischemia (Shimohama, S. (1998) Brain Res. 779, 359-363), trauma (Socci, D. J., Arendash, G. W. (1996) Mol. Chem. Neuropathol. 27, 285-305), AB-mediated neuronal death (Rusted, J. M., et al. (2000) Behav. Brain Res. 113, 121-129; Kihara, T., et al. (1997) Ann. Neurol. 42, 159-163; Kihara, T., et al. (2001) J. Biol. Chem. 276, 13541-13546) and protein-aggregation mediated neuronal degeneration (Kelton, M. C. et al.(2000) Brain Cogn 43, 274-282). In many instances where nicotine displays a neuroprotective effect, a direct involvement of receptors comprising the α7 subtype has been invoked (Shimohama, S. et al. (1998) Brain Res. 779, 359-363; Kihara, T., et al. (2001) J. Biol. Chem. 276, 13541-13546; Kelton, M. C., et al. (2000) Brain Cogn 43, 274-282; Kem, W. R. (2000) Behav. Brain Res. 113, 169-181; Dajas-Bailador, F. A., et al. (2000) Neuropharmacology 39, 2799-2807; Strahlendorf, J. C., et al. (2001) Brain Res. 901, 71-78) suggesting that activation of α7 subtype-containing nicotinic acetylcholine receptors may be instrumental in mediating the neuroprotective effects of nicotine. Available data suggest that the α7 nicotinic acetylcholine receptor represents a valid molecular target for the development of agonists/positive modulators active as neuroprotective molecules. Indeed, α7 nicotinic receptor agonists have already been identified and evaluated as possible leads for the development of neuroprotective drugs (Jonnala, R. R., et al.(2002) Life Sci. 70, 1543-1554; Bencherif, M., et al. (2000) Eur. J. Pharmacol. 409, 45-55; Donnelly-Roberts, D. L., et al. (1996) Brain Res. 719, 36-44; Meyer, E. M., et al. (1998) J. Pharmacol. Exp. Ther. 284, 1026-1032; Stevens, T. R., et al. (2003) J. Neuroscience 23, 10093-10099). Compounds described herein can be used to treat such diseases.

In accordance with the invention, there is provided a method of treating a patient, especially a human, suffering from age-related dementia and other dementias and conditions with memory loss comprising administering to the patient an effective amount of a compound according to Formula I.

The present invention includes methods of treating patients suffering from memory impairment due to, for example, mild cognitive impairment due to aging, Alzheimer's disease, schizophrenia, Parkinson's disease, Huntington's disease, Pick's disease, Creutzfeldt-Jakob disease, depression, aging, head trauma, stroke, CNS hypoxia, cerebral senility, multiinfarct dementia and other neurological conditions, as well as HIV and cardiovascular diseases, comprising administering an effective amount of a compound according to Formula I.

In accordance with an embodiment of the invention there is provided a method of treating and/or preventing dementia in an Alzheimer's patient which comprises administering to the subject a therapeutically effective amount of a compound according to Formula I to inhibit the binding of an amyloid beta peptide (preferably, Aβ1-42) with nACh receptors, preferable α7nACh receptors, most preferably, human α7nACh receptors (as well as a method for treating and/or preventing other clinical manifestations of Alzheimer's disease that include, but are not limited to, cognitive and language deficits, apraxias, depression, delusions and other neuropsychiatric symptoms and signs, and movement and gait abnormalities).

The present invention also provides methods for treating other amyloidosis diseases, for example, hereditary cerebral angiopathy, nonneuropathic hereditary amyloid, Down's syndrome, macroglobulinemia, secondary familial Mediterranean fever, Muckle-Wells syndrome, multiple myeloma, pancreatic- and cardiac-related amyloidosis, chronic hemodialysis anthropathy, and Finnish and Iowa amyloidosis.

In addition, nicotinic receptors have been implicated as playing a role in the body's response to alcohol ingestion. Thus, agonists for α7nACh receptors can be used in the treatment of alcohol withdrawal and in anti-intoxication therapy. Thus, in accordance with an embodiment of the invention there is provided a method of treating a patient for alcohol withdrawal or treating a patient with anti-intoxication therapy comprising administering to the patient an effective amount of a compound according to formula I.

Agonists for the α7nACh receptor subtypes can also be used for neuroprotection against damage associated with strokes and ischemia and glutamate-induced excitotoxicity. Thus, in accordance with an embodiment of the invention there is provided a method of treating a patient to provide for neuroprotection against damage associated with strokes and ischemia and glutamate-induced excitotoxicity comprising administering to the patient an effective amount of a compound according to formula I.

Agonists for the α7nACh receptor subtypes can also be used in the treatment of nicotine addiction, inducing smoking cessation, treating pain, and treating jetlag, obesity, diabetes, sexual and fertility disorders (eg. Premature ejaculation or vaginal dryness, see U.S. Pat. No. 6,448,276), drug abuse (Solinas, Journal of Neuroscience (2007) 27(21), 5615-5620), and inflammation (Wang H, et al. (2003) Nature 421 :384-388). Thus, in accordance with an embodiment of the invention there is provided a method of treating a patient suffering from nicotine addiction, drug abuse, pain, jetlag, obesity and/or diabetes, or a method of inducing smoking cessation in a patient comprising administering to the patient an effective amount of a compound according to Formula I.

The inflammatory reflex is an autonomic nervous system response to an inflammatory signal. Upon sensing an inflammatory stimulus, the autonomic nervous system responds through the vagus nerve by releasing acetylcholine and activating nicotinic α7 receptors on macrophages. These macrophages in turn release cytokines. Dysfunctions in this pathway have been linked to human inflammatory diseases including rheumatoid arthritis, diabetes and sepsis. Macrophages express the nicotinic α7 receptor and it is likely this receptor that mediates the cholinergic anti-inflammatory response. Therefore, compounds with affinity for the α7nACh receptor on macrophages may be useful for human inflammatory diseases including rheumatoid arthritis, diabetes and sepsis. See, e.g., Czura, C J et al., J. Intern. Med., (2005) 257(2), 156-66, Wang, H. et al Nature (2003) 421: 384-388; de Jonge British Journal of Pharmacology (2007) 151, 915-929.

Thus, in accordance with an embodiment of the invention there is provided a method of treating a patient (e.g., a mammal, such as a human) suffering from an inflammatory disease, such as, but not limited to, rheumatoid arthritis, diabetes or sepsis, comprising administering to the patient an effective amount of a compound according to formula I.

The mammalian sperm acrosome reaction is an exocytosis process important in fertilization of the ovum by sperm. Activation of an α7 nAChR on the sperm cell has been shown to be essential for the acrosome reaction (Son, J.-H. and Meizel, S. Biol, Reproduct. 68: 1348-1353 2003). Consequently, selective α7 agents demonstrate utility for treating fertility disorders.

In addition, due to their affinity to α7nACh receptors, labeled derivatives of the compounds of formula I (for example Cl I or F18 labeled derivatives), can be used in neuroimaging of the receptors within, e.g., the brain. Thus, using such labeled agents in vivo imaging of the receptors can be performed using, for example PET imaging.

The condition of memory impairment is manifested by impairment of the ability to learn new information and/or the inability to recall previously learned information. Memory impairment is a primary symptom of dementia and can also be a symptom associated with such diseases as Alzheimer's disease, schizophrenia, Parkinson's disease, Huntington's disease, Pick's disease, Creutzfeldt-Jakob disease, HIV, cardiovascular disease, and head trauma as well as age-related cognitive decline.

Thus, in accordance with an embodiment of the invention there is provided a method of treating a patient suffering from, for example, mild cognitive impairment (MCI), vascular dementia (VaD), age-associated cognitive decline (AACD), amnesia associated w/open-heart-surgery, cardiac arrest, and/or general anesthesia, memory deficits from early exposure of anesthetic agents, sleep deprivation induced cognitive impairment, chronic fatigue syndrome, narcolepsy, AIDS-related dementia, epilepsy-related cognitive impairment, Down's syndrome, Alcoholism related dementia (Korsakoff Syndrome), drug/substance induced memory impairments, Dementia Puglistica (Boxer Syndrome), and animal dementia (e.g., dogs, cats, horses, etc.) comprising administering to the patient an effective amount of a compound according to formula I.

Dosage of the compounds for use in therapy may vary depending upon, for example, the administration route, the nature and severity of the disease. In general, an acceptable pharmacological effect in humans may be obtained with daily dosages ranging from 0.01 to 200 mg/kg.

In some embodiments of the present invention, one or more compounds of formula I are administered in combination with one or more other pharmaceutically active agents. The phrase “in combination”, as used herein, refers to agents that are simultaneously administered to a subject. It will be appreciated that two or more agents are considered to be administered “in combination” whenever a subject is simultaneously exposed to both (or more) of the agents. Each of the two or more agents may be administered according to a different schedule; it is not required that individual doses of different agents be administered at the same time, or in the same composition. Rather, so long as both (or more) agents remain in the subject's body, they are considered to be administered “in combination”.

For example, compounds of formula I, in forms as described herein, may be administered in combination with one or more other modulators of α7 nicotinic acetylcholine receptors. Alternatively or additionally, compounds of formula I, in forms as described herein, may be administered in combination with one or more other anti-psychotic agents, pain relievers, anti-inflammatories, or other pharmaceutically active agents.

Effective amounts of a wide range of other pharmaceutically active agents are well known to those skilled in the art. However, it is well within the skilled artisan's purview to determine the other pharmaceutically active agent's optimal effective amount range. The compound of formula I and the other pharmaceutically active agent can act additively or, in some embodiments, synergistically. In some embodiments of the invention, where another pharmaceutically active agent is administered to an animal, the effective amount of the compound of formula I is less than its effective amount would be where the other pharmaceutically active agent is not administered. In this case, without wishing to be bound by any particular theory, it is believed that a compound of formula I and the other pharmaceutically active agent act synergistically. In some cases, the patient in need of treatment is being treated with one or more other pharmaceutically active agents. In some cases, the patient in need of treatment is being treated with at least two other pharmaceutically active agents.

In some embodiments, the other pharmaceutically active agent is selected from the group consisting of one or more anti-depressant agents, anti-anxiety agents, anti-psychotic agents, and cognitive enhancers. Examples of classes of antidepressants that can be used in combination with the active compounds of this invention include norepinephrine reuptake inhibitors, selective serotonin reuptake inhibitors (SSRIs), NK-1 receptor antagonists, monoamine oxidase inhibitors (MAOs), reversible inhibitors of monoamine oxidase (RIMAs), serotonin and noradrenaline reuptake inhibitors (SNRIs), corticotropin releasing factor (CRF) antagonists, α-adrenoreceptor antagonists, and atypical antidepressants. Suitable norepinephrine reuptake inhibitors include tertiary amine tricyclics and secondary amine tricyclics. Suitable tertiary amine tricyclics and secondary amine tricyclics include amitriptyline, clomipramine, doxepin, imipramine, trimipramine, dothiepin, butriptyline, iprindole, lofepramine, nortriptyline, protriptyline, amoxapine, desipramine and maprotiline. Suitable selective serotonin reuptake inhibitors include fluoxetine, citolopram, escitalopram, fluvoxamine, paroxetine and sertraline. Examples of monoamine oxidase inhibitors include isocarboxazid, phenelzine, and tranylcypromine. Suitable reversible inhibitors of monoamine oxidase include moclobemide. Suitable serotonin and noradrenaline reuptake inhibitors of use in the present invention include venlafaxine, nefazodone, milnacipran, and duloxetine. Suitable CRF antagonists include those compounds described in International Patent Publication Nos. WO 94/13643, WO 94/13644, WO 94/13661, WO 94/13676 and WO 94/13677. Suitable atypical anti-depressants include bupropion, lithium, nefazodone, trazodone and viloxazine. Suitable NK-1 receptor antagonists include those referred to in International Patent Publication WO 01/77100.

Anti-anxiety agents that can be used in combination with the compounds of formula I include without limitation benzodiazepines and serotonin 1A (5-HT_{1A}) agonists or antagonists, especially 5-HT_{1A} partial agonists, and corticotropin releasing factor (CRF) antagonists. Exemplary suitable benzodiazepines include alprazolam, chlordiazepoxide, clonazepam, chlorazepate, diazepam, halazepam, lorazepam, oxazepam, and prazepam. Exemplary suitable 5-HT_{1A} receptor agonists or antagonists include buspirone, flesinoxan, gepirone and ipsapirone.

Anti-psychotic agents that are used in combination with the compounds of formula I include without limitation aliphatic phethiazine, a piperazine phenothiazine, a butyrophenone, a substituted benzamide, and a thioxanthine. Additional examples of such drugs include without limitation haloperidol, olanzapine, clozapine, risperidone, pimozide, aripiprazol, and ziprasidone. In some cases, the drug is an anticonvulsant, e.g., phenobarbital, phenytoin, primidone, or carbamazepine.

Cognitive enhancers that are used in combination with the compounds of formula I include, without limitation, drugs that modulate neurotransmitter levels (e.g., acetylcholinesterase or cholinesterase inhibitors, cholinergic receptor agonists or serotonin receptor antagonists), drugs that modulate the level of soluble Aβ3, amyloid fibril formation, or amyloid plaque burden (e.g., y-secretase inhibitors, β-secretase inhibitors, antibody therapies, and degradative enzymes), and drugs that protect neuronal integrity (e.g., antioxidants, kinase inhibitors, caspase inhibitors, and hormones). Other representative candidate drugs that are co-administered with the compounds of the invention include cholinesterase inhibitors, (e.g., tacrine (COGNEX®), donepezil (ARICEPT®), rivastigmine (EXELON®) galantamine (REMINYL®), metrifonate, physostigmine, and Huperzine A), N-methyl-D-aspartate (NMDA) antagonists and agonists (e.g., dextromethorphan, memantine, dizocilpine maleate (MK-801), xenon, remacemide, eliprodil, amantadine, D-cycloserine, felbamate, ifenprodil, CP-101606 (Pfizer), Delucemine, and compounds described in U.S. Pat. Nos. 6,821,985 and 6,635,270), ampakines (e.g., cyclothiazide, aniracetam, CX-516 (Ampalex®), CX-717, CX-516, CX-614, and CX-691 (Cortex Pharmaceuticals, Inc. Irvine, Calif.), 7-chloro-3-methyl-3-4-dihydro-2H-1,2,4-benzothiadiazine S,S-dioxide (see Zivkovic et al., 1995, J. Pharmacol. Exp. Therap., 272:300-309; Thompson et al., 1995, Proc. Natl. Acad. Sci. USA, 92:7667-7671), 3-bicyclo[2,2,1]hept-5-en-2-yl-6-chloro-3,4-dihydro-2H-1,2,4-benzothiadiazine-7-sulfonamide-1,1-dioxide (Yamada, et al., 1993, J. Neurosc. 13:3904-3915); 7-fluoro-3-methyl-5-ethyl-1,2,4-benzothiadiazine-S,S-dioxide; and compounds described in U.S. Pat. No. 6,620,808 and International Patent Application Nos. WO 94/02475, WO 96/38414, WO 97/36907, WO 99/51240, and WO 99/42456), benzodiazepine (BZD)/GABA receptor complex modulators (e.g., progabide, gengabine, zaleplon, and compounds described in U.S. Pat. No. 5,538,956, 5,260,331, and 5,422,355); serotonin antagonists (e.g., 5HT receptor modulators, 5HT_{1A} antagonists or agonists (including without limitation lecozotan and compounds described in U.S. Pat. Nos. 6,465,482, 6,127,357, 6,469,007, and 6,586,436, and in PCT Publication No. WO 97/03982) and 5-HT₆ antagonists (including without limitation compounds described in U.S. Pat. Nos. 6,727,236, 6,825,212, 6,995,176, and 7,041,695)); nicotinics (e.g., niacin); muscarinics (e.g., xanomeline, CDD-0102, cevimeline, talsaclidine, oxybutin, tolterodine, propiverine, tropsium chloride and darifenacin); monoamine oxidase type B (MAO B) inhibitors (e.g., rasagiline, selegiline, deprenyl, lazabemide, safinamide, clorgyline, pargyline, N-(2-aminoethyl)-4-chlorobenzamide hydrochloride, and N-(2-aminoethyl)-5(3-fluorophenyl)-4-thiazolecarboxamide hydrochloride); phosphodiesterase (PDE) IV inhibitors (e.g., roflumilast, arofylline, cilomilast, rolipram, RO-20-1724, theophylline, denbufylline, ARIFLO, ROFLUMILAST, CDP-840 (a tri-aryl ethane) CP80633 (a pyrimidone), RP 73401 (Rhone-Poulenc Rorer), denbufylline (SmithKline Beecham), arofylline (Almirall), CP-77,059 (Pfizer), pyrid[2,3d]pyridazin-5-ones (Syntex), EP-685479 (Bayer), T-440 (Tanabe Seiyaku), and SDZ-ISQ-844 (Novartis)); G proteins; channel modulators; immunotherapeutics (e.g., compounds described in U.S. Patent Application Publication No. US 2005/0197356 and US 2005/0197379); anti-amyloid or amyloid lowering agents (e.g., bapineuzumab and compounds described in U.S. Pat. No. 6,878,742 or U.S. Patent Application Publication Nos. US 2005/0282825 or US 2005/0282826); statins and peroxisome proliferators activated receptor (PPARS) modulators (e.g., gemfibrozil (LOPID®), fenofibrate (TRICOR®), rosiglitazone maleate (AVANDIA®), pioglitazone (Actos™), rosiglitazone (Avandia™), clofibrate and bezafibrate); cysteinyl protease inhibitors; an inhibitor of receptor for advanced glycation endproduct (RAGE) (e.g., aminoguanidine, pyridoxaminem carnosine, phenazinediamine, OPB-9195, and tenilsetam); direct or indirect neurotropic agents (e.g., Cerebrolysin®, piracetam, oxiracetam, AIT-082 (Emilieu, 2000, Arch. Neurol. 57:454)); beta-secretase (BACE) inhibitors, a-secretase, immunophilins, caspase-3 inhibitors, Src kinase inhibitors, tissue plasminogen activator (TPA) activators, AMPA (alpha-amino-3-hydroxy-5-methyl-4-isoxazolepropionic acid) modulators, M4 agonists, JNK3 inhibitors, LXR agonists, H3 antagonists, and angiotensin IV antagonists. Other cognition enhancers include, without limitation, acetyl-1-carnitine, citicholine, huperzine, DMAE (dimethylaminoethanol), Bacopa monneiri extract, Sage extract, L-alpha glyceryl phosphoryl choline, Ginko biloba and Ginko biloba extract, Vinpocetine, DHA, nootropics including Phenyltropin, Pikatropin (from Creative Compounds, LLC, Scott City, Mo.), besipirdine, linopirdine, sibopirdine, estrogen and estrogenic compounds, idebenone, T-588 (Toyama Chemical, Japan), and FK960 (Fujisawa Pharmaceutical Co. Ltd.). Compounds described in U.S. Pat. Nos. 5,219,857, 4,904,658, 4,624,954 and 4,665,183 are also useful as cognitive enhancers as described herein. Cognitive enhancers that act through one or more of the above mechanisms are also within the scope of this invention.

In some embodiments, the compound of formula I and cognitive enhancer act additively or, in some embodiments, synergistically. In some embodiments, where a cognitive enhancer and a compound of formula I of the invention are co-administered to an animal, the effective amount of the compound or pharmaceutically acceptable salt of the compound of the invention is less than its effective amount would be where the cognitive enhancer agent is not administered. In some embodiments, where a cognitive enhancer and a compound of formula I are co-administered to an animal, the effective amount of the cognitive enhancer is less than its effective amount would be where the compound or pharmaceutically acceptable salt of the invention is not administered. In some embodiments, a cognitive enhancer and a compound of formula I of the invention are co-administered to an animal in doses that are less than their effective amounts would be where they were not co-administered. In these cases, without wishing to be bound by any particular theory, it is believed that the compound of formula I and the cognitive enhancer act synergistically.

In some embodiments, the other pharmaceutically active agent is an agent useful for treating Alzheimer's disease or conditions associate with Alzheimer's disease, such as dementia. Exemplary agents useful for treating Alzheimer's disease include, without limitation, donepezil, rivastigmine, galantamine, memantine, and tacrine.

In some embodiments, the compound of formula I is administered together with another pharmaceutically active agent in a single administration or composition.

In some embodiments, a composition comprising an effective amount of the compound of formula I and an effective amount of another pharmaceutically active agent within the same composition can be administered.

In some embodiments, a composition comprising an effective amount of the compound of formula I and a separate composition comprising an effective amount of another pharmaceutically active agent can be concurrently administered. In some embodiments, an effective amount of the compound of formula I is administered prior to or subsequent to administration of an effective amount of another pharmaceutically active agent. In certain embodiments, the compound of formula I is administered while the other pharmaceutically active agent exerts its therapeutic effect, or the other pharmaceutically active agent is administered while the compound of formula I exerts its preventative or therapeutic effect.

Thus, in some embodiments, the invention provides a composition comprising an effective amount of the compound of formula I of the present invention and a pharmaceutically acceptable carrier. In some embodiments, the composition further comprises a second pharmaceutically active agent.

In some embodiments, the composition further comprises a pharmaceutically active agent selected from the group consisting of one or more other antidepressants, anti-anxiety agents, anti-psychotic agents or cognitive enhancers. Antidepressants, anti-anxiety agents, anti-psychotic agents and cognitive enhancers suitable for use in the composition include the antidepressants, anti-anxiety agents, anti-psychotic agents and cognitive enhancers provided above.

In some embodiments, the pharmaceutically acceptable carrier is suitable for oral administration and the composition comprises an oral dosage form.

In some embodiments, one or more compounds of formula I are administered in combination with antidepressant drug treatment, antipsychotic drug treatment, and/or anticonvulsant drug treatment.

In certain embodiments, a compound of formula I is administered in combination with one or more selective serotonin reuptake inhibitors (SSRIs) (for example, fluoxetine, citalopram, escitalopram oxalate, fluvoxamine maleate, paroxetine, or sertraline), tricyclic antidepressants (for example, desipramine, amitriptyline, amoxipine, clomipramine, doxepin, imipramine, nortriptyline, protriptyline, trimipramine, dothiepin, butriptyline, iprindole, or lofepramine), aminoketone class compounds (for example, bupropion); in some embodiments, a compound of formula I is administered in combination with a monoamine oxidase inhibitor (MAOI) (for example, phenelzine, isocarboxazid, or tranylcypromine), a serotonin and norepinepherine reuptake inhibitor (SNRI) (for example, venlafaxine, nefazodone, milnacipran, duloxetine), a norepinephrine reuptake inhibitor (NRI) (for example, reboxetine), a partial 5-HT_{1A} agonist (for example, buspirone), a 5-HT_{2A} receptor antagonist (for example, nefazodone), a typical antipsychotic drug, or an atypical antipsychotic drug. Examples of such antipsychotic drugs include aliphatic phethiazine, a piperazine phenothiazine, a butyrophenone, a substituted benzamide, and a thioxanthine. Additional examples of such drugs include haloperidol, olanzapine, clozapine, risperidone, pimozide, aripiprazol, and ziprasidone. In some cases, the drug is an anticonvulsant, e.g., phenobarbital, phenytoin, primidone, or carbamazepine. In some cases, the compound of formula I is administered in combination with at least two drugs that are antidepressant drugs, antipsychotic drugs, anticonvulsant drugs, or a combination thereof.

### Pharmaceutical Compositions

In yet a further aspect, the invention refers to a pharmaceutical composition containing one or more compounds of formula I, in association with pharmaceutically acceptable carriers and excipients. The pharmaceutical compositions can be in the form of solid, semi-solid or liquid preparations, preferably in form of solutions, suspensions, powders, granules, tablets, capsules, syrups, suppositories, aerosols or controlled delivery systems. The compositions can be administered by a variety of routes, including oral, transdermal, subcutaneous, intravenous, intramuscular, rectal and intranasal, and are preferably formulated in unit dosage form, each dosage containing from about 1 to about 1000 mg, preferably from 1 to 600 mg of the active ingredient. The compounds of the invention can be in the form of free bases or as acid addition salts, preferably salts with pharmaceutically acceptable acids. The invention also includes separated isomers and diastereomers of compounds of formula I, or mixtures thereof (e.g. racemic mixtures). The principles and methods for the preparation of pharmaceutical compositions are described for example in Remington's Pharmaceutical Science, Mack Publishing Company, Easton (Pa.).

When administered to an animal, one or more compounds of formula I, in any desirable form (e.g., salt form, crystal form, etc.), can be administered neat or as a component of a pharmaceutical composition that comprises a physiologically acceptable carrier or vehicle. Such a pharmaceutical composition of the invention can be prepared using standard methods, for example admixing the compound(s) and a physiologically acceptable carrier, excipient, or diluent. Admixing can be accomplished using methods well known for admixing a compound of formula I and a physiologically acceptable carrier, excipient, or diluent.

Provided pharmaceutical compositions (i.e., comprising one or more compounds of formula I, in an appropriate form, can be administered orally. Alternatively or additionally, provided pharmaceutical compositions can be administered by any other convenient route, for example, parenterally (e.g., subcutaneously, intravenously, etc., by infusion or bolus injection, etc), by absorption through epithelial or mucocutaneous linings (e.g., oral, rectal, vaginal, and intestinal mucosa, etc.), etc. Administration can be systemic or local. Various known delivery systems, including, for example, encapsulation in liposomes, microparticles, microcapsules, and capsules, can be used.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. In some instances, administration will result of release of the compound (and/or one or more metabolites thereof) into the bloodstream. The mode of administration may be left to the discretion of the practitioner.

In some embodiments, provided pharmaceutical compositions are administered orally; in some embodiments, provided pharmaceutical compositions are administered intravenously.

In some embodiments, it may be desirable to administer provided pharmaceutical compositions locally. This can be achieved, for example, by local infusion during surgery, topical application, e.g., in conjunction with a wound dressing after surgery, by injection, by means of a catheter, by means of a suppository or edema, or by means of an implant, said implant being of a porous, non-porous, or gelatinous material, including membranes, such as sialastic membranes, or fibers.

In certain embodiments, it can be desirable to introduce a compound of formula I into the central nervous system, circulatory system or gastrointestinal tract by any suitable route, including intraventricular, intrathecal injection, paraspinal injection, epidural injection, enema, and by injection adjacent to the peripheral nerve. Intraventricular injection can be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, the compound of formula I can be formulated as a suppository, with traditional binders and excipients such as triglycerides.

In some embodiments, one or more compounds of formula I can be delivered in a vesicle, in particular a liposome (see Langer, Science 249:1527-1533, 1990 and Treat et al., Liposomes in the Therapy of Infectious Disease and Cancer 317-327 and 353-365, 1989).

In some embodiments, one or more compounds of formula I can be delivered in a controlled-release system or sustained-release system (see, e.g., Goodson, in Medical Applications of Controlled Release, vol. 2, pp. 115-138, 1984). Other controlled or sustained-release systems discussed in the review by Langer, Science 249:1527-1533, 1990 can be used. In some embodiments, a pump can be used (Langer, Science 249:1527-1533, 1990; Sefton, CRC Crit. Ref Biomed. Eng. 14:201, 1987; Buchwald et al., Surgery 88:507, 1980; and Saudek et al., N. Engl. J Med. 321:574, 1989). In another embodiment, polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 2:61, 1983; Levy et al., Science 228:190, 1935; During et al., Ann. Neural. 25:351, 1989; and Howard et al., J. Neurosurg. 71:105, 1989).

As noted above, provided pharmaceutical compositions can optionally comprise a suitable amount of a physiologically acceptable excipient. Exemplary physiologically acceptable excipients can be liquids, such as water and oils, including those of petroleum, animal, vegetable, or synthetic origin, such as peanut oil, soybean oil, mineral oil, sesame oil and the like. For example, useful physiologically acceptable excipients can be saline, gum acacia, gelatin, starch paste, talc, keratin, colloidal silica, urea and the like. Alternatively or additionally, auxiliary, stabilizing, thickening, lubricating, and coloring agents can be used.

In some embodiments, a physiologically acceptable excipient that is sterile when administered to an animal is utilized. Such physiologically acceptable excipients are desirably stable under the conditions of manufacture and storage and will typically be preserved against the contaminating action of microorganisms. Water is a particularly useful excipient when a compound of formula I is administered intravenously. Saline solutions and aqueous dextrose and glycerol solutions can also be employed as liquid excipients, particularly for injectable solutions. Suitable physiologically acceptable excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Provided pharmaceutical compositions, if desired, can also contain minor amounts of wetting or emulsifying agents, or pH buffering agents.

Liquid carriers may be used in preparing solutions, suspensions, emulsions, syrups, and elixirs. A compound of formula I can be dissolved or suspended in a pharmaceutically acceptable liquid carrier such as water, an organic solvent, a mixture of both, or pharmaceutically acceptable oils or fat. Such a liquid carrier can contain other suitable pharmaceutical additives including solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particularly containing additives as above, e.g., cellulose derivatives, including sodium carboxymethyl cellulose solution), alcohols (including monohydric alcohols and polyhydric alcohols, e.g., glycols) and their derivatives, and oils (e.g., fractionated coconut oil and arachis oil). For parenteral administration the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

Provided pharmaceutical compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In some embodiments, pharmaceutical compositions in the form of a capsule are provided. Other examples of suitable physiologically acceptable excipients are described in Remington 's Pharmaceutical Sciences 1447-1676 (Alfonso R. Gennaro, ed., 19th ed. 1995).

In some embodiments, a compound of formula I (in an appropriate form) is formulated in accordance with routine procedures as a composition adapted for oral administration to humans. Compositions for oral delivery can be in the form of tablets, lozenges, buccal forms, troches, aqueous or oily suspensions or solutions, granules, powders, emulsions, capsules, syrups, or elixirs, for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. In powders, the carrier can be a finely divided solid, which is an admixture with the finely divided compound or pharmaceutically acceptable salt of the compound. In tablets, the compound or pharmaceutically acceptable salt of the compound is mixed with a carrier having the necessary compression properties in suitable proportions and compacted in the shape and size desired. The powders and tablets can contain up to about 99% of the compound or pharmaceutically acceptable salt of the compound.

Capsules may contain mixtures of one or more compounds of formula I with inert fillers and/or diluents such as pharmaceutically acceptable starches (e.g., corn, potato, or tapioca starch), sugars, artificial sweetening agents, powdered celluloses (such as crystalline and microcrystalline celluloses), flours, gelatins, gums, etc.

Tablet formulations can be made by conventional compression, wet granulation, or dry granulation methods and utilize pharmaceutically acceptable diluents, binding agents, lubricants, disintegrants, surface modifying agents (including surfactants), suspending or stabilizing agents (including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins.) Surface modifying agents include nonionic and anionic surface modifying agents. Representative examples of surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecylsulfate, magnesium aluminum silicate, and triethanolamine.

Moreover, when in a tablet or pill form, provided pharmaceutical compositions can be coated to delay disintegration and absorption in the gastrointestinal tract, thereby providing a sustained action over an extended period of time. Selectively permeable membranes surrounding an osmotically active driving compound are also suitable for orally administered compositions. In these latter platforms, fluid from the environment surrounding the capsule can be imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In some embodiments, the excipients are of pharmaceutical grade.

In some embodiments, one or more compounds of formula I (in an appropriate form) can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lidocaine to lessen pain at the site of the injection. Generally, the ingredients are supplied either separately or mixed together in unit dosage form, for example, as a dry lyophilized powder or water-free concentrate in a hermetically sealed container such as an ampule or sachette indicating the quantity of active agent. Where a compound of formula I is to be administered by infusion, it can be dispensed, for example, with an infusion bottle containing sterile pharmaceutical grade water or saline. Where a compound of formula I is administered by injection, an ampule of sterile water for injection or saline can be provided so that the ingredients can be mixed prior to administration.

In some embodiments, one or more compounds of formula I (in an appropriate form) can be administered transdermally through the use of a transdermal patch. Transdermal administrations include administrations across the surface of the body and the inner linings of the bodily passages including epithelial and mucosal tissues. Such administrations can be carried out using the present in lotions, creams, foams, patches, suspensions, solutions, and suppositories (e.g., rectal or vaginal).

Transdermal administration can be accomplished through the use of a transdermal patch containing one or more compounds of formula I (in an appropriate form) and a carrier that is inert to the compound or pharmaceutically acceptable salt of the compound, is non-toxic to the skin, and allows delivery of the agent for systemic absorption into the blood stream via the skin. The carrier may take any number of forms such as creams or ointments, pastes, gels, or occlusive devices. The creams or ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the compound or pharmaceutically acceptable salt of the compound into the blood stream, such as a semi-permeable membrane covering a reservoir containing a compound of formula I with or without a carrier, or a matrix containing the active ingredient.

One or more compounds of formula I (in an appropriate form) may be administered rectally or vaginally in the form of a conventional suppository. Suppository formulations may be made from traditional materials, including cocoa butter, with or without the addition of waxes to alter the suppository's melting point, and glycerin. Water-soluble suppository bases, such as polyethylene glycols of various molecular weights, may also be used.

One or more compounds of formula I (in an appropriate form) can be administered by controlled-release or sustained-release means or by delivery devices that are known to those of ordinary skill in the art. Such dosage forms can be used to provide controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled- or sustained-release.

In some embodiments a controlled- or sustained-release composition comprises a minimal amount of a compound of formula I to treat or prevent one or more disorders, diseases or conditions associated with activity of α7 nicotinic acetylcholine receptors. Advantages of controlled- or sustained-release compositions include extended activity of the drug, reduced dosage frequency, and increased compliance by the subject being treated. In addition, controlled- or sustained-release compositions can favorably affect the time of onset of action or other characteristics, such as blood levels of the compound or a pharmaceutically acceptable salt of the compound, and can thus reduce the occurrence of adverse side effects.

Controlled- or sustained-release compositions can initially release an amount of one or more compounds of formula I that promptly produces a desired therapeutic or prophylactic effect, and gradually and continually release other amounts of the compound to maintain this level of therapeutic or prophylactic effect over an extended period of time. To maintain a constant level of the compound a body, the compound can be released from the dosage form at a rate that will replace the amount of the compound being metabolized and excreted from the body. Controlled- or sustained-release of an active ingredient can be stimulated by various conditions, including but not limited to, changes in pH, changes in temperature, concentration or availability of enzymes, concentration or availability of water, or other physiological conditions or compounds.

In certain embodiments, provided pharmaceutical compositions deliver an amount of a compound of formula I that is effective in the treatment of one or more disorders, diseases, or conditions associated with activity (or inactivity) of α7 nicotinic acetylcholine receptors. According to the present invention, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy will be determined according to the judgment of a health-care practitioner. Effective dosage amounts described herein typically refer to total amounts administered; that is, if more than one compound of formula I is administered, the effective dosage amounts correspond to the total amount administered.

The effective amount of a compound of formula I for use as described herein will typically range from about 0.001 mg/kg to about 600 mg/kg of body weight per day, in some embodiments, from about 1 mg/kg to about 600 mg/kg body weight per day, in some embodiments, from about 10 mg/kg to about 400 mg/kg body weight per day, in some embodiments, from about 10 mg/kg to about 200 mg/kg of body weight per day, in some embodiments, from about 10 mg/kg to about 100 mg/kg of body weight per day, in some embodiments, from about 1 mg/kg to about 10 mg/kg body weight per day, in some embodiments, from about 0.001 mg/kg to about 100 mg/kg of body weight per day, in some embodiments, from about 0.001 mg/kg to about 10 mg/kg of body weight per day, and in some embodiments, from about 0.001 mg/kg to about 1 mg/kg of body weight per day.

In some embodiments, pharmaceutical compositions are provided in unit dosage form, e.g., as a tablet, capsule, powder, solution, suspension, emulsion, granule, or suppository. In such form, the composition is sub-divided in unit dose containing appropriate quantities of the active ingredient; the unit dosage form can be packaged compositions, for example, packeted powders, vials, ampoules, prefilled syringes or sachets containing liquids. A unit dosage form can be, for example, a capsule or tablet itself, or it can be the appropriate number of any such compositions in package form. Such unit dosage form may contain, for example, from about OO1 mg/kg to about 250 mg/kg, and may be given in a single dose or in two or more divided doses. Variations in the dosage will necessarily occur depending upon the species, weight and condition of the patient being treated and the patient's individual response to the medicament.

In some embodiments, the unit dosage form is about 0.01 to about 1000 mg. In another embodiment, the unit dosage form is about 0.01 to about 500 mg; in another embodiment, the unit dosage form is about 0.01 to about 250 mg; in another embodiment, the unit dosage form is about 0.01 to about 100 mg; in another embodiment, the unit dosage form is about 0.01 to about 50 mg; in another embodiment, the unit dosage form is about 0.01 to about 25 mg; in another embodiment, the unit dosage form is about 0.01 to about 10 mg; in another embodiment, the unit dosage form is about 0.01 to about 5 mg; and in another embodiment, the unit dosage form is about 0.01 to about 10 mg;

A compound of formula I can be assayed in vitro or in vivo for the desired therapeutic or prophylactic activity prior to use in humans. Animal model systems can be used to demonstrate safety and efficacy.

### Exemplification

The compounds of the invention can be prepared through a number of synthetic routes amongst which the ones illustrated in Schemes 1-4 below:

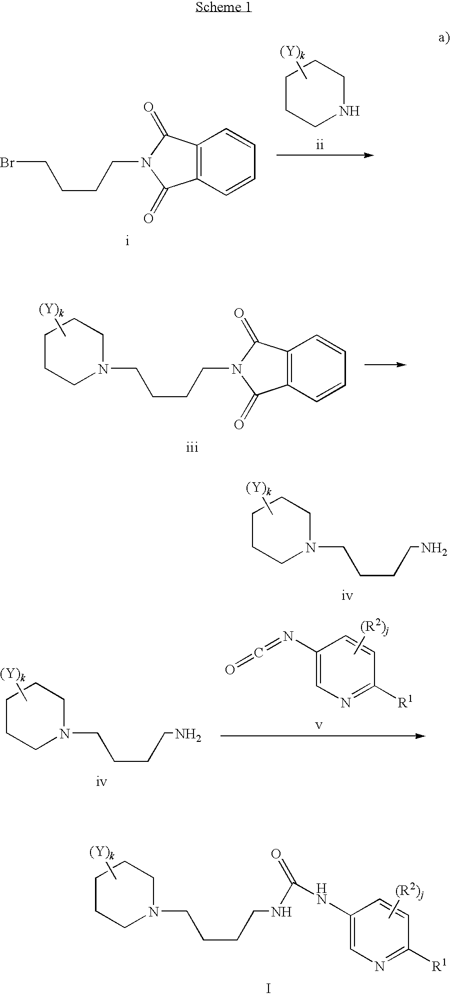

According to Scheme 1, a suitably activated 4-halobutylphthalimide i is reacted with a piperidine ii in an organic solvent such as 2-butanone or dimethylformamide in the presence of a base such as triethylamine or potassium carbonate. For example, a mixture of ii (or its hydrochloride salt) and i are refluxed in methylethyl ketone in the presence of alkaline carbonate until the reaction is complete, then the reaction mixture is cooled, the insoluble materials removed by filtration, the filtrate washed with chloroform or dichloromethane, and the filtrate and washings concentrated to dryness.

In the following step, 4-piperidinobutylphthalimide iii is converted into a 4-piperidinobutylamine iv, for example by refluxing a mixture of iii and hydrazine hydrate in ethanol. Diamine iv is then reacted with an activated species v such as for example an isocyanate or equivalent (for example a carbamoyl chloride or a reactive carbamate such a vinyl or aryl carbamate), hereby exemplified by an arylisocyanate in an organic solvent such as dichloromethane, tetrahydrofuran, dimethylformamide or mixtures thereof, to give compounds of formula I.

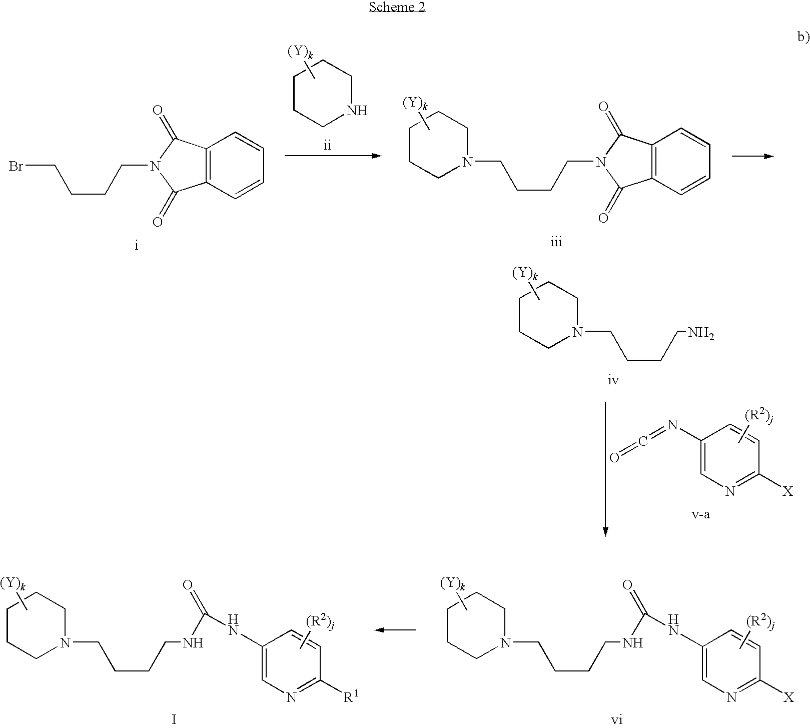

Scheme 2 essentially follows Scheme 1, with the difference that diamine iv is reacted with an activated species v-a such as for example an isocyanate or equivalent (for example a carbamoyl chloride or a reactive carbamate such a vinyl or aryl carbamate) to give a further intermediate vi where X is a leaving group, for example an halogen or a mesylate. Intermediate vi is then reacted under carbon-carbon coupling conditions, such as for example Suzuki coupling in a solvent such as for example tetrahydrofuran, dichloroethane, acetonitrile, dimethylformamide, water or mixtures of the formers and the like, which may necessitate thermal of microwave heating and transition metal catalysis, to give compounds of formula I.

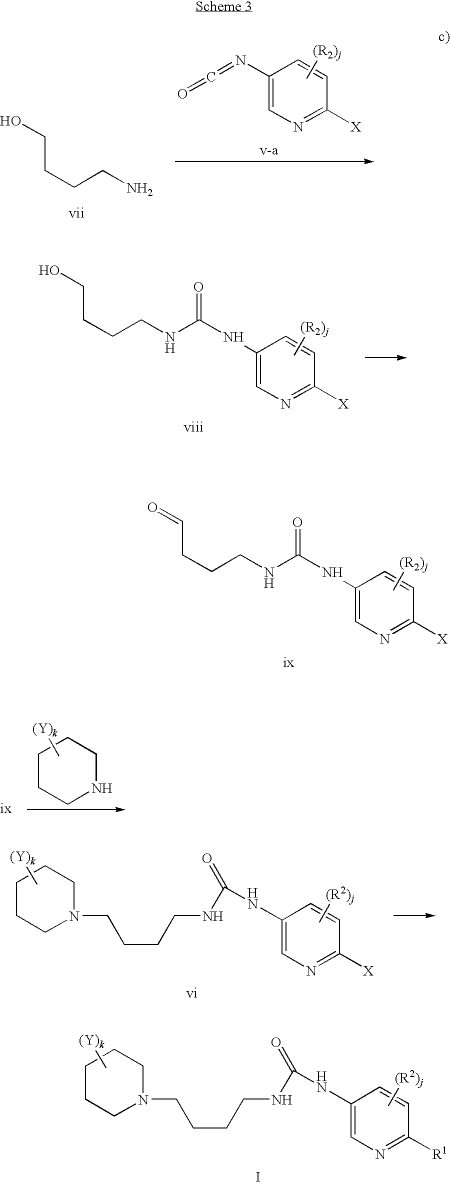

According to Scheme 3, a 4-hydroxybutylamine is reacted with an isocyanate or equivalent (for example a carbamoyl chloride or a reactive carbamate such as a vinyl or aryl carbamate), hereby exemplified by an arylisocyanate, in an organic solvent such as for example dichloromethane, tetrahydrofuran, dimethylformamide or mixtures thereof, until the reaction is complete. The ureidobutanol viii thus obtained is then oxidised under standard conditions (for example Swern oxidation) and aldehyde ix is then reacted with a piperidine under standard reductive alkylation conditions—for example with sodium triacetoxyborohydride—to afford compound vi, which in the case of X being R¹ gives compounds of formula I. In the case of X being a halogen or a boronic acid ester, compound 5 can be further processed—for example via a cross-coupling reaction, with a boronic acid or an aryl or heteroaryl halide, for example under the conditions of the Suzuki coupling, which may necessitate thermal of microwave heating and transition metal catalysis, to afford compounds of formula I.

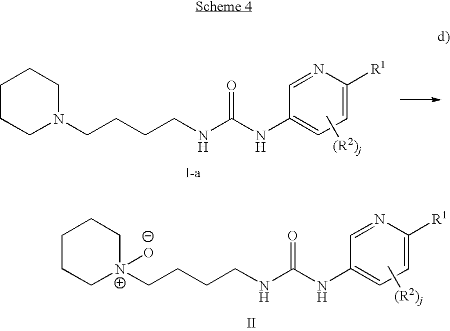

According to Scheme 4, a compound of formula I-a is reacted under oxidative conditions, for example by treating is with a peroxyacid such as 3-chloroperoxybenzoic acid or a peroxyphthalate salt or for example hydrogen peroxide in the presence or absence of a carboxylic acid, in a solvent such as (for example but not limited to) dichloromethane or methanol, to afford N-oxide compounds of formula II.

### EXAMPLES

### Experimental Procedures—Synthesis of Compounds

### General

Unless otherwise specified all nuclear magnetic resonance spectra were recorded using a Varian Mercury Plus 400 MHz spectrometer equipped with a PFG ATB Broadband probe.

HPLC-MS analyses were performed with a Waters 2795 separation module equipped with a Waters Micromass ZQ (ES ionisation) and Waters PDA 2996, using a Waters XTerra MS Cl8 3.5 μm 2.1×50 mm column.

Preparative HLPC was run using a Waters 2767 system with a binary Gradient Module Waters 2525 pump and coupled to a Waters Micromass ZQ (ES) or Waters 2487 DAD, using a Supelco Discovery HS C18 5.0 μm 10×21.2 mm column

Gradients were run using 0.1% formic acid/water and 0.1% formic acid/acetonitrile with gradient 5/95 to 95/5 in the run time indicated.

All column chromatography was performed following the method of Still, C.; J. Org Chem 43, 2923 (1978). All TLC analyses were performed on silica gel (Merck 60 F254) and spots revealed by UV visualisation at 254 nm and KMnO4 or ninhydrin stain.

When specified for array synthesis, heating was performed on a Buchi Syncore® system.

All microwave reactions were performed in a CEM Discover oven.

### Abbreviations Used Throughout the Experimental Procedures

DCM dichloromethane
DCE 1,2-dichloroethane
DMEA N,N-dimethylethylamine
DMF N,N-dimethylformamide
DMSO, dmso dimethylsulphoxide
DAM N,N-dimethylacetamide
Scx strong cation exchanger
TEA triethylamine
TFA trifluoroacetic acid
THF tetrahydrofuran
TLC thin layer chromatography
LC-MS Liquid chromatography—mass spectrometry
HPLC High performance (pressure) liquid chromatography

### 4-Piperidin-1-yl-butylamine

Prepared following a modification of the general procedure outlined in Nishikawa, Y.; et al; Chem. Pharm. Bull., 1989, 37 (1), 100-105;

### a) 2-(4-Piperidin-1-yl-butl)-isoindole-1,3-dione

To a solution of piperidine (58 mL, 0.587 mol, 1.6 equiv.) in 2-butanone (2.5 L), NaI (61.6 g, 0.411 mol, 0.74 equiv.), K₂CO₃ (122 g, 0.88 mol, 1.6 equiv.) and N-(4-bromobutyl)phthalimide (156 g, 0.533 mol, 1 equiv.) were added. The reaction mixture was refluxed (internal temperature=82° C.) under stirring for 18 hours.

The mixture was cooled at room temperature and the inorganic salts filtered off and washed with EtOAc. The organic phase was washed with brine (2.5 L), dried and concentrated to afford an off-white solid that was washed with isopropyl alcohol to afford 2-(4-piperidin-1-ylbutyl)-1H-isoindole-1,3(2H)-dione as a white solid (124.88 g).

C₁₇H₂₂N₂O₂ Mass (calculated) [286.38]; (found) [M+H+]=287

LC Rt=0.97, 95% (5 min method)

NMR (400 MHz, CDCl₃) 1.41 (2H, m), 1.49-1.59 (6H, m), 1.65-1.72 (2H, m), 2.15-2.35 (6H, m), 3.69-3.73 (6H, m), 7.69-7.74 (2H, m), 7.80-7.85 (2H, m)

FTIR (KBr):λmax 3455, 2933, 2766, 1700, 1464, 1436, 1399, 1369, 1258, 1228, 1103, 1044, 925, 866, 719, 529 cm⁻¹.

### b) 4-Piperidin-1-yl-butylamine

Hydrazine monohydrate (80 mL, 1.65 mol, 3.6 equiv.) was added dropwise to a solution of intermediate 2-(4-piperidin-1-ylbutyl)-1H-isoindole-1,3(2H)-dione (130 g, 0.454 mol, 1 equiv.) in EtOH (3.1 L) and the mixture was refluxed (internal temperature=80° C.) for about 4 hours. The reaction mixture was cooled at room temperature, the insoluble phthalhydrazide filtered off and the filtrate evaporated under reduced pressure. The obtained oil was dissolved in CH₂Cl₂ and filtered to remove some more phthalhydrazide (this procedure was repeated until complete disappearance of phthalhydrazide was observed). The filtrate was concentrated to give 1-(4-butylamino)piperidine as a yellow oil (66 g).

C₉H₂₀N₂ Mass (calculated) [156.27]; (found) [M+H+]=157

LC Rt=0.31 (5 min method)

NMR (400 MHz, CD₃OD): 1.45-1.62 (10 H, m), 2.30-2.43 (10 H, m), 2.64-2.67 (2H, m)

FTIR (KBr):λmax 3358, 2933, 1572, 1471, 1383, 1310, 1155, 1121, 1039, 780 cm⁻¹.

### 1-(6-Bromo-pyridin-3-yl)-3-(4-piperidin-1-yl-butyl)-urea

To a solution of 6-bromo-pyridin-3-ylamine (3.46 g, 20 mmol, 1 equiv.) in DCM (70 mL) triphosgene (1.96 g, 6.6 mmol, 0.33 equiv.) and TEA (2.2 g, 22 mmol, 1.1 equiv.) were added under N₂ at 0° C. After 15 min a solution of 4-piperidin-1-yl-butylamine (3.12 g, 20 mmol, 1 equiv.) in DCM (10 mL) was added dropwise at 0° C. The reaction mixture was allowed to warm to room temperature and stirred for 2 h. Dichloromethane was removed in vacuo and the residue dissolved in EtOAc and washed with H₂O. The aqueous layer was basified with solid Na₂CO₃ to pH 9-10 and extracted with EtOAc. The combined organic layers were dried over Na₂SO₄ and evaporated in vacuo to give 5 g of the desired product as a solid (yield: 70%).

C₁₅H₂₃BrN₄O Mass (calculated) [355]

¹H-NMR (400 MHz, CDCl₃): 8.17 (d, 1H, J=2.8), 7.95 (dd, 1H, J=8.7, 2.8), 7.54 (bs, 1H), 7.35 (d, 1H, J=8.7), 6.19 (m, 1H), 3.24-3.22 (m, 2H), 2.48-2.29 (m, 8H), 1.62-1.45 (m, 8H).

### Suzuki Coupling Procedure—General Method A

To a degassed solution of 1-(6-bromo-pyridin-3-yl)-3-(4-piperidin-1-yl-butyl)-urea (107 mg, 0.3 mmol, 1 equiv.) in toluene (2 mL), a degassed solution of the appropriate boronic acid (0.45 mmol, 1.5 equiv.) in EtOH (1 mL) and Cs₂CO₃ (195 mg, 0.6 mmol, 2 equiv.) were added followed by Pd[(PPh₃)]₄ (18 mg, 0.015 mmol, 0.05 equiv.). The solution was irradiated under microwave conditions, using the following parameters: power=200 watt; ramp time=1 min; hold time=20 min; temp=90° C.; pressure=200 psi. The solvents mixture was removed in vacuo and the crude mixture was purified using a SCX column washing with dichloromethane/MeOH followed by MeOH and then NH₃/MeOH to elute the product. The fractions containing the desired product were combined and dried under reduced pressure. The crude was washed with Et₂O to obtain the desired product.

### Suzuki Coupling Procedure—General Method B

To a degassed suspension of 1-(6-bromo-pyridin-3-yl)-3-(4-piperidin-1-yl-butyl)-urea (100 mg, 0.28 mmol, 1 equiv.) in CH₃CN (1 mL), the appropriate boronic acid (0.34 mmol, 1.2 equiv.), a solution of 0.4 M Na₂CO₃ (1 mL) and Pd[(PPh₃)]₄ (16 mg, 0.01 mmol, 0.05 equiv.) were added. The solution was irradiated under microwave conditions, using the following parameters: power=200 watt; ramp time=1 min; hold time=10 min; temp=90° C.; pressure=200 psi. To the reaction mixture ethyl acetate (1 mL) was added and the resulting organic layer was pipetted out and put on top of a SCX cartridge (2 g). The crude mixture was then worked-up washing the SCX cartridge with MeOH and then a solution of NH₃ in 7 N MeOH to elute the product. The fractions containing the desired product were combined and dried under reduced pressure. The crude was purified using prep-HPLC.

### Suzuki Coupling Procedure—General Method C

To a degassed mixture of 1-(6-bromo-pyridin-3-yl)-3-(4-piperidin-1-yl-butyl)-urea (0.1 g, 1 equiv.), the appropriate boronic acid or ester (1.2 equiv.) in acetonitrile/sodium carbonate 0.4 M solution 1/1 (2 mL) and a catalytic amount of Pd[(PPh₃)]₄ (5 mmol %) was added. The reaction mixture was heated at 60° C. overnight. The organic layer was separated, filtered and evaporated. The residue was dissolved in CH₃CN/H₂O and purified by preparative HPLC.

### Example 1

### 1-[6-(2-Fluoro-5-methoxy-phenyl)-pyridin-3-yl]-3-(4-piperidin-1-yl-butyl)-urea

The product was prepared according to procedure A

Yield: 83%

C₂₂H₂₉FN₄O₂ Mass (calculated) [400]; (found) [M+H⁺]=401

LC Rt=1.69, 100% (10 min method)

¹H-NMR (400 MHz, CD₃OD): 8.64 (d, 1H, J=2.6), 7.97 (dd, 1H, J=8.7, 2.6), 7.68 (dd, 1H, J=8.7, 2.1), 7.32 (m, 1H), 7.09 (m, 1H), 6.91 (m, 1H), 3.81 (s, 3H), 3.24-3.21 (m, 2H), 2.42-2.32 (m, 6H), 1.62-1.45 (m, 10H)

### Example 2

### 1-(6-Benzo[1,3]dioxol-5-yl-pyridin-3-yl)-3-(4-piperidin-1-yl-butyl)-urea

The product was prepared according to procedure A

Yield: 77%

C₂₂H₂₈N₄O₃ Mass (calculated) [396]; (found) [M+H+]=397

LC Rt=1.31, 100% (10 min method)

¹H-NMR (400 MHz, CD₃OD): 8.52 (d, 1H, J=2.6), 7.93 (dd, 1H, J=8.6, 2.6), 7.67 (d, 1H, J=8.6), 7.37-7.36 (m, 2H), 6.87 (m, 1H), 5.98 (m, 2H), 3.24-3.20 (m, 2H), 2.43-2.33 (m, 6H), 1.61-1.45 (m, 10H).

### Example 3

### 1-[6-(2-Fluoro-phenyl)-pyridin-3-yl]-3-(4-piperidin-1-yl-butyl)-urea

The product was prepared according to procedure A

Yield: 72%

C₂₁H₂₇FN₄O Mass (calculated) [370]; (found) [M+H⁺]=371

LC Rt=1.54, 99% (10 min method)

¹H-NMR (400 MHz, CD₃OD): 8.59 (dd, 1H, J=2.6, 0.6), 7.99 (dd, 1H, J=8.7, 2.6), 7.75 (dd, 1H, J=8.7, 0.6), 7.69 (m, 1H), 7.64 (m, 1H), 7.43 (m, 1H), 7.08 (m, 1H), 3.24-3.20 (m, 2H), 2.41-2.31 (m, 6H), 1.61-1.44 (m, 10H).

### Example 4

### 1-(4-Piperidin-1-yl-butyl)-3-(6-thiophen-3-yl-pyridin-3-yl)-urea

The product was prepared according to procedure A

Yield: 74%

C₁₉H₂₆N₄OS Mass (calculated) [358]; (found) [M+H⁺]=359

LC Rt=0.99, 96% (10 min method)

¹H-NMR (400 MHz, CD₃OD): 8.45 (d,1H, J=2.6), 7.86 (dd, 1H, J=8.7, 2.6), 7.78 (dd, 1H, J=3.0, 1.3), 7.62 (d, 1H, J=8.7), 7.53 (dd, 1H, J=5.1, 1.3), 7.39 (dd, 1H, J=5.1, 3.0), 3.17-3.14 (m, 2H), 2.42-2.32 (m, 6H), 1.57-1.40 (m, 10H).

### Example 5

### 1-[2,4′]Bipyridinyl-5-yl-3-(4-piperidin-1-yl-butyl)-urea

The product was prepared according to procedure A

Yield: 12%

C₂₀H₂₇N₅O Mass (calculated) [353]; (found) [M+H⁺]=354

LC Rt=0.59, 100% (10 min method)

¹H-NMR (400 MHz, CD₃OD): 8.59 (d, 1H, J=2.6), 8.51-8.50 (m, 2H), 7.99 (dd, 1H, J=8.7, 2.6), 7.91-7.89 (m, 2H), 7.85 (d, 1H, J=8.7), 3.18-3.15 (m, 2H), 2.40-2.29 (m, 6H), 1.56-1.39 (m, 10H).

### Example 6

### 1-(4-Piperidin-1-yl-butyl)-3-(6-thiophen-2-yl-pyridin-3-yl)-urea formic acid salt

The product was prepared according to procedure A. Further purification was done by prep-HPLC to obtain 47 mg of the title compound.

Yield: 44%

C₁₉H₂₆N₄OS.HCOOH Mass (calculated) [358.46]; (found) [M+H⁺]=359

LC Rt=1.49, 100% (10 min method)

¹H-NMR (400 MHz, DMSO): 9.29 (bs, 1H), 8.50 (m, 1H), 8.31 (s, 1H), 7.94 (d, 1H, J=8.7), 7.76 (d, 1H, J=8.7), 7.60 (m, 1H), 7.50 (m, 1H), 7.10 (m, 1H), 6.88 (bs, 1H), 3.11-3.06 (m, 2H), 2.68-2.58 (m, 6H), 1.59-1.44 (m, 10H).

### Example 7

### 1-[6-(1-Methyl-1H-pyrazol-4-yl)-pyridin-3-yl]-3-(4-piperidin-1-yl-butyl)-urea formic acid salt

The product was prepared according to procedure B and further purified by preparative HPLC.

Yield: 28%

C₁₉H₂₈N₆O.HCOOH Mass (calculated) [356.46]; (found) [M+H⁺]=357

LC Rt=0.56, 100% (10 min method)

¹H-NMR (400 MHz, DMSO): 9.56 (bs, 1H), 9.26 (bs, 1H), 8.65 (bs, 1H), 8.32 (bs, 1H), 8.32 (bs, 1H), 8.12 (s, 1H), 8.01-7.71 (m, 2H), 6.67 (bs, 1H), 3.87 (s, 3H), 3.44-3.25 (m, 2H), 3.15-2.98 (m, 4H), 2.87-2.79 (m, 2H), 1.80-1.33 (m, 10H).

### Example 8

### 1-(6′-Methoxy-[2,3′]bipyridinyl-5-yl)-3-(4-piperidin-1-yl-butyl)-urea formic acid salt

The product was prepared according to procedure B and further purified by preparative HPLC.

Yield: 64%

C₂₁H₂₉N₅O₂HCOOH Mass (calculated) [383.46]; (found) [M+H⁺]=384

LC Rt=1.31, 100% (10 min method)

¹H-NMR (400 MHz, DMSO): 9.09 (s, 1H), 8.76 (d, 1H, J=2.5), 8.60 (d, 1H, J=2.6), 8.29-8.26 (m, 2H), 7.97 (dd, 1H, J=8.7, 2.6), 7.80 (d, 1H, J=8.7), 6.87 (d, 1H, J=8.7), 8.69 (m, 1H), 3.88 (s, 3H), 3.12-3.07 (m, 2H), 2.65-2.50 (m, 6H), 1.60-1.30 (m, 10H).

### Example 9

### 1-(6′-Fluoro-[2,3′]bipyridinyl-5-yl)-3-(4-piperidin-1-yl-butyl)-urea formic acid salt

The product was prepared according to procedure B and further purified by preparative HPLC.

Yield: 41%

C₂₀H₂₆FN₅O.HCOOH Mass (calculated) [371.46]; (found) [M+H⁺]=372

LC Rt=1.34, 100% (10 min method)

¹H-NMR (400 MHz, DMSO): 9.19 (s, 1H), 8.82 (d, 1H, J=2.5), 8.65 (d, 1H, J=2.5), 8.53 (m, 1H), 8.26 (s, 1H), 8.02 (dd, 1H, J=8.7, 2.6), 7.90 (d, 1H, J=8.6), 7. 24 (dd, 1H, J=8.6, 2.8), 6.75 (m, 1H), 3.12-3.07 (m, 2H), 2.65-2.47 (m, 6H), 1.57-1.36 (m, 10H).

### Example 10

### 1-[6-(2-Fluoro-phenyl)-pyridin-3-yl]-3-(4-piperidin-1-yl-butyl)-urea formic acid salt

The product was prepared according to procedure B

Yield: 55%

C₂₁H₂₇FN₄O.HCOOH Mass (calculated) [370.46]; (found) [M+H⁺]=371

LC Rt=1.41, 100% (10 min method)

¹H-NMR (400 MHz, DMSO): 9.16 (s, 1H), 8.68 (d, 1H), 8.23 (s, 1H), 7.97 (dd, 1H), 8.89 (m, 1H), 7.66 (dd, 1H), 7.39 (m, 1H), 7.30-7.25 (m, 2H), 6.69 (m, 1H), 3.13-3.08 (m, 2H), 2.70-2.61 (m, 6H), 1.62-1.40 (m, 10H).

### Example 11

### 1-(4-Piperidin-1-yl-butyl)-3-[6-(1H-pyrazol-4-yl)-pyridin-3-yl]-ureaformic acid salt

The product was prepared according to procedure B. After a first irradiation under microwave conditions, another cycle of irradiation using the same conditions and an extra addition of Pd[(PPh₃)]₄ (0.05 equiv.) were necessary to complete the reaction. Purification was achieved by preparative HPLC.

Yield: 40%

C₁₈H₂₆N₆O.HCOOH Mass (calculated) [342.46]; (found) [M+H⁺]=343

LC Rt=0.36, 100% (10 min method)

¹H-NMR (400 MHz, DMSO): 8.86 (s, 1H), 8.45 (d, 1H, J=2.6), 8.18 (s, 1H), 8.04 (bs, 2H), 7.84 (dd, 1H, J=8.6, 2.6), 8.77 (s, 1H), 7.53 (d, 1H, J=8.6), 6.50 (m, 1H), 3.12-3.07 (m, 2H), 2.88-2.71 (m, 6H), 1.67-1.55 (m, 6H), 1.47-1.39 (m, 4H).

### Example 12

### 1-(6-Furan-2-yl-pyridin-3-yl)-3-(4-piperidin-1-yl-butyl)-urea formic acid salt

The product was prepared according to procedure B and further purified by preparative HPLC.

Yield: 50%

C₁₉H₂₆N₄O₂.HCOOH Mass (calculated) [342.46]; (found) [M+H⁺]=343

LC Rt=1.06, 100% (10 min method)

¹H-NMR (400 MHz, DMSO): 9.10 (s, 1H), 8.53 (d, 1H, J=2.5), 8.24 (s, 1H), 7.95 (dd, 1H, J=8.7, 2.5), 7.73 (m, 1H), 7.58 (d, 1H, J=8.7), 6.89 (m, 1H), 6.66 (m, 1H), 6.58 (m, 1H), 3.11-3.07 (m, 2H), 2.70-2.54 (m, 6H), 1.60-1.49 (m, 6H), 1.46-1.39 (m, 4H).

### Example 13

### 1-(6-Phenyl-pyridin-3-yl)-3-(4-piperidin-1-yl-butyl)-urea

To a degassed solution of 1-(6-bromo-pyridin-3-yl)-3-(4-piperidin-1-yl-butyl)-urea (2 g, 5.63 mmol, 1 equiv.), phenylboronic acid (0.82 g, 6.75 mmol, 1.2 equiv.) and 0.4 M Na₂CO₃ (15 mL) in CH₃CN (30 mL), Pd[(PPh₃)]₄ (50 mg, 0.04 mmol, 0.007 equiv.) was added The reaction mixture was stirred at 90° C. for 7 hours. After 7 hours an extra amount of phenylboronic acid (1 equiv.) and Pd[(PPh₃)]₄ (0.007 equiv.) was added due to suboptimal conversion. The reaction mixture was stirred for a further 7 hours at 90° C., followed by an addition of phenylboronic acid (1 equiv.) and Pd[(PPh₃)]₄ (0.007 equiv.). The reaction mixture was stirred for a further 8 hours at 120° C. LCMS showed still low conversion into the product, so the reaction mixture was filtered to eliminate insoluble residues and fresh phenylboronic acid (1 equiv.) and Pd[(PPh₃)]₄ (0.007 equiv.) were added again. The reaction mixture was stirred again for 24 hours at 120° C.

The CH₃CN was removed in vacuo and the aqueous crude solution was basified with solid Na₂CO₃ then extracted with ethyl acetate (3×40 mL). The aqueous layer was basified again with Na₂CO₃ to pH>10 and extracted with ethyl acetate (2×40 mL). The combined organic phases were evaporated in vacuo to give a solid corresponding to the title product.

A pure sample (0.56 g) was obtained by crystallisation from an ethyl acetate/diisopropyl ether (1:1) mixture. The mother liquor was evaporated in vacuo and the residue purified using a SCX cartridge (10 g) washing with MeOH and then eluting with a 7 N NH₃ methanolic solution. The fractions containing the desired product were combined and dried under reduced pressure to give an additional 0.38 g of 1-(6-phenyl-pyridin-3-yl)-3-(4-piperidin-1-yl-butyl)-urea.

Yield: 47%

C₂₁H₂₈N₄O Mass (calculated) [352]; (found) [M+H⁺]=353

LC Rt=1.21, 99% (10 min method)

¹H-NMR (400 MHz, DMSO): 8.67 (s, 1H), 8.57 (d, 1H), 8.00-7.96 (m, 3H), 7.82 (d, 1H), 7.45-7.41 (m, 2H), 7.34 (m, 1H), 6.30 (m, 1H), 3.11-3.06 (m, 2H), 2.33-2.18 (m, 6H), 1.49-1.32 (m, 10H).

### 1-(6-Phenyl-pyridin-3-yl)-3-(4-piperidin-1-yl-butyl)-urea hydrochloric salt

To a solution of 1-(6-Phenyl-pyridin-3-yl)-3-(4-piperidin-1-yl-butyl)-urea (916 mg, 2.6 mmol, 1 equiv.) in MeOH (10 mL) a solution of 2 M HCl in Et₂O (4 mL, 3.9 mmol, 1.5 equiv.) was added and the reaction mixture stirred at room temperature for 4 hours. The product was isolated by filtration.

Yield: 100%

C₂₁H₂₈N₄O.HCl Mass (calculated) [352.36]; (found) [M+H⁺]=353

LC Rt=1.21, 99% (10 min method)

¹H-NMR (400 MHz, DMSO): 10.40 (bs, 1H), 10.22 (bs, 1H), 9.03 (s, 1H), 8.27-8.21 (m, 2H), 8.06-8.00 (m, 2H), 7.60-7.53 (m, 3H), 7.09 (bs, 1H), 3.38-3.35 (m, 2H), 3.20-3.10(m, 2H), 3.04-2.95 (m, 2H), 2.85-2.76 (m, 2H), 1.80-1.65 (m, 7H), 1.50-1.30 (m, 3H).

### Example 14

### 1-[2,3′]Bipyridinyl-5-yl-3-(4-piperidin-1-yl-butyl)-urea

Pyridine-3-boronic acid (42 mg, 0.312 mmol, 1.2 equivalents) was weighed into a clean microwave vessel and dissolved in acetonitrile (1 mL). To this, 1-(6-bromo-pyridin-3-yl)-3-(4-piperidin-1-yl-butyl)-urea was added (100 mg, 0.262 mmol), along with tetrakis(triphenylphosphine)palladium (20 mg, 0.017 mmol) and a solution of sodium carbonate (1 mL, 0.4 M). The reaction mixture was then exposed to microwave irradiation at psi 250, 90° C. for 20 minutes. On reaction completion by LCMS analysis, the extracted crude organic phase was filtered through a plug of Celite® and washed through with dichloromethane. The collected sample was loaded onto a Si column (2 g) and the column washed with a solution of dichloromethane/methanol (1-20% methanol gradient) to remove impurities. Washing with a solution of ammonia in methanol (20% ammonia) afforded the desired product 1-[2,3′]bipyridinyl-5-yl-3-(4-piperidin-1-yl-butyl)-urea dried to yield (10.2 mg, 0.029 mmol, 11%) as a solid.

C₂₀H₂₇N₅O Mass (calculated) [353.47]; (found) [M+H⁺]=354

LC Rt=Double peak at solvent front observed at 0.23, 0.48-0.91 90% (10 min method)

NMR (400 MHz, DMSO-d6): 1.62-1.74 (6H, m); 2.37-2.61 (10H, m); 3.31 (2H, s); 6.55 (1H, s); 7.39 (2H, m); 7.68 (1H, m); 8.15-8.23 (2H, m); 8.54-8.59 (2H, m); 9.14 (1H, s).

### Example 15

### 1-[6-(2-Methoxyphenyl)-pyridin-3-yl]-3-(4-piperidin-1-yl-butyl)-urea

1-(6-Bromo-pyridin-3-yl)-3-(4-piperidin-1-yl-butyl)-urea was weighed into a microwave vessel (100 mg, 0.28 mmol) and dissolved in acetonitrile (1 mL). To this, 2-methoxyphenylboronic acid (52 mg, 0.34 mmol) was added, along with tetrakis(triphenylphosphine)palladium (20 mg, 0.017 mmol) and a solution of sodium carbonate (1 mL, 0.4 M). The reaction mixture was then exposed to microwave irradiation at psi 250, 90° C. for 20 minutes. On reaction completion by LCMS analysis, the separated organic phase was removed from the reaction mix and passed through a plug of Celite®. The collected crude was treated with a solution of acetonitrile/water (3: 1) from which the desired product crystallised. The solid was collected, washed with diethyl ether and dried to yield the titled compound (21 mg, 0.055 mmol, 19.6% yield)

C₂₂H₃₀N₄O₂ Mass (calculated) [382.51]; (found) [M+H⁺]=383

LC Rt=Double peak observed at 0.23 and 1.14 90% (10 min method)

NMR (400 MHz, CDCl₃): 1.46-1.68 (10H, m); 2.32-2.38 (6H, m); 3.26 (2H, m); 3.81 (3H, s); 6.09 (1H, s); 6.49 (1H, s); 6.97-7.05 (2H, m); 7.26-7.34 (1H, m); 7.71 (2H, m); 7.99-8.02 (1H, m) 8.45 (1H, s).

### Example 16

### 1-[6-(4-Methoxy-phenyl)-pyridin-3-yl]-3-(4-piperidin-1-yl-butyl)-urea

1-(6-Bromo-pyridin-3-yl)-3-(4-piperidin-1-yl-butyl)-urea was weighed into a microwave vessel (100 mg, 0.28 mmol) and dissolved in acetonitrile (1 mL). To this, 4-methoxyphenylboronic acid (52 mg, 0.34 mmol) was added, along with tetrakis(triphenylphosphine)palladium (20 mg, 0.017 mmol) and a solution of sodium carbonate (1 mL, 0.4 M). The reaction mixture was then exposed to microwave irradiation at psi 250, 90° C. for 20 minutes. On reaction completion by LCMS analysis, the separated organic phase was removed from the reaction mixture and passed through a plug of Celite®. The collected crude was treated with a solution of acetonitrile/water (3:1) from which the desired product crystallized. The solid was collected, washed with ether and dried to yield the titled compound (36 mg, 0.094 mmol, 33.7% yield)

C₂₂H₃₀N₄O₂ Mass (calculated) [382.51]; (found) [M+H⁺]=383

LC Rt=Double peak at solvent front observed at 0.23 and 1.23 98% (10 min method)

NMR (400 MHz, CDCl₃): 1.46 (2H, m); 1.58 (6H, m); 1.76 (2H, s); 2.31-2.38 (6H, m); 3.26 (2H, m); 3.85 (3H, s); 6.14 (1H, m); 6.55 (1H, m); 6.95-6.98 (2H, m); 7.59-7.61 (1H, m); 7.86 (2H, m); 8.03-8.05 (1H, m) 8.38 (1H, s).

### Example 17

### 1-[6-(4-Fluoro-phenyl)-pyridin-3-yl]-3-(4-piperidin-1-yl-butyl)-urea hydrochloride

### a) (6-Bromo-pyridin-3-yl)-carbamic acid isopropenyl ester

To a solution of NaOH (1.13 g, 28.3 mmol) in 56 mL of water, a solution of 5-amino-2-bromopyridine (3.26 g, 18.8 mmol) in 112 mL of DCM was added. The mixture was cooled at 0° C. and isopropenyl chloroformate (3.16 g, 2.84 mL, 26.4 mmol) was added in one hour dissolved in 15 mL of DCM maintaining the solution at 0° C.

The mixture was then allowed to reach room temperature and stirred overnight. The organic phase was separated and evaporated at reduced pressure maintaining the temperature of the evaporator bath below 25° C.

The crude product obtained was used in the next reaction without further purification.

C₉H₉BrN₂O₂ calculated 257; found M+257-259

Lc Rt (5 min)=1.88

LC Area % (215 nm)=84%

### b) 1-(6-Bromo-pyridin-3-yl)-3-(4-piperidin-1-yl-butyl)-urea

The crude product obtained in the previous reaction was dissolved in 80 mL of THF and 4-piperidin-1-yl-butylamine (2.94 g, 18.8 mmol) was added. The solution was refluxed under nitrogen atmosphere for 2.5 hours. After evaporation of the solvent, the product was dissolved in DCM. The organic phase was washed with brine, evaporated and dried. 6.216 g of product was obtained (yield: 93%).

C₁₅H₂₃BrN₄O calculated 355; found M+355-357

Lc Rt (5 min)=1.08

LC Area % (215 nm)=100%

### c) 1-[6-(4-Fluoro-phenyl)-pyridin-3-yl]-3-(4-piperidin-1-yl-butyl)-urea

1-(6-Bromo-pyridin-3-yl)-3-(4-piperidin-1-yl-butyl)-urea (6.216 g, 17.51 mmol), 4-fluorophenylboronic acid (3.677 g, 26.26 mmol) and cesium carbonate (11.41 g, 35.02 mmol) were dissolved in 143 mL of Toluene and 72 mL of EtOH and the mixture degassed with a nitrogen stream.

Tetrakis(triphenylphosphine)palladium (0.607 g, 0.52 mmol) was added and the mixture heated at 90° C. for 2 h.

The warm reaction mixture was then filtered on Celite® and the solvent evaporated. The product was purified by SiO₂ column (gradient from 100% DCM to DCM-NH₃ 2 N in methanol 8:2)

The product obtained was crystallised from ethyl acetate affording 2.138 g (yield: 33%) of the title product.

C₂₁H₂₇FN₄O calculated 370; found M+371

Lc Rt (10 min)=1.75

LC Area % (215 nm)=99%

¹H-NMR (400 MHz, DMSO): 1.31-1.47 (10H, m); 22.19-2.34 (6H, m); 3.07-3.09 (2H, m); 6.31 (1H, t, J=5.6); 7.25 (2H, t, J=8.9); 7.81 (1H, d, J=8.7); 7.95-8.04 (3H, m), 8.56 (1H, d, J=2.5); 8.68 (1H, s).

### d) 1-[6-(4-Fluoro-phenyl)-pyridin-3-yl]-3-(4-piperidin-1-yl-butyl)-urea hydrochloride

1-[6-(4-Fluoro-phenyl)-pyridin-3-yl]-3-(4-piperidin-1-yl-butyl) urea free base (2.108 g, 5.70 mmol) was suspended in 100 mL of DCM and 1.2 eq of HCl (2 M in Et₂O) was added. The solvent was then evaporated and the product washed with Et₂O to obtain 1-[6-(4-fluoro-phenyl)-pyridin-3-yl]-3-(4-piperidin-1-yl-butyl)-urea hydrochloric acid salt (1.851 g, yield: 80%).

C₂₁H₂₇FN₄O calculated 370; found M+371

Lc Rt (10 min)=1.62

LC Area % (215 nm)=100%

¹H-NMR (400 MHz, DMSO): 1.30-1.78 (10H, m); 2.76-2.85 (2H, m); 2.97-3.03 (2H, m); 3.11-3.13 (2H, m); 3.36-3.39 (2H, m); 6.80 (1H, s); 7.31 (2H, t, J=8.9); 7.94 (1H, d, J=8.8); 8.02-8.06 (3H, m); 8.74 (1H, d, J=2.2); 9.56 (1H, s); 9.94 (1H, s).

### Example 18

### 1-[6-(4-Fluoro-phenyl)-pyridin-3-yl]-3-[4-(1-oxy-piperidin-1-yl)-butyl]-urea

1-[6-(4-Fluoro-phenyl)-pyridin-3-yl]-3-(4-piperidin-1-yl-butyl)-urea (1.274 g, 3.44 mmol) was dissolved in DCM and 3-chloroperoxybenzoic acid (70% aq; 0.846 g, 3.44 mmol) was added and the solution was stirred until no starting material was present. The organic solvent was then evaporated and the crude product dissolved in methanol. The byproduct 3-chloro-benzoic acid was eliminated by filtration on a Isolute SPE NH₂ column eluting with methanol.

After evaporation of the solvent 1.152 g (yield: 87%) of pure product was obtained.

C₂₁H₂₇FN₄O₂ calculated 386; found M+387

Lc Rt (10 min)=1.52

LC Area % (215 nm)=100%

¹H-NMR (400 MHz, CD₃OD): 1.42-1.73 (6H, m); 1.88-1.96 (2H, m); 2.08-2.18 (2H, m); 3.18-3.31 (8H, m); 7.17 (2H, t, J=8.8); 7.73 (1H, d, J=12.5); 7.89-7.92 (2H, m), 7.98 (1H, dd, J=8.7, J=2.7); 8.59 (1H, d, J=3.2).

### Example 19

### 1-(2′-Methoxy-[2,3′]bipyridinyl-5-yl)-3-(4-piperidin-1-yl-butyl)-urea

Prepared via Suzuki Coupling procedure—according to General Method C, to give 79.6 mg (74%) of title compound

C₂₁H₂₉N₅O₂ Mass (calculated) [383.5]; found [M+H⁺]=384.3

Lc RT=0.23 and 1.14 (double peak, 10 min method), 100%

NMR (400 MHz, CD₃OD): 1.57-1.69 (4H, m); 1.73-1.89 (6H, m); 3.07-3.12 (2H, m), 3.26-3.30 (6H, m), 3.98 (3H, s), 7.08 (1H, dd, J=4.98 Hz, J=7.44 Hz), 7.84 (1H, d, J=8.69 Hz), 7.93 (1H, dd, J=2.62 Hz, J=8.69 Hz), 8.04 (1H, dd, J=1.93 Hz, J=7.44 Hz), 8.17 (1H, dd, J=1.93, Hz, J=4.98 Hz), 8.53 (1H, s), 8.66 (1H, d, J=2.62 Hz).

### Example 20

### 1-(6′-Chloro-[2,3′]bipyridinyl-5-yl)-3-(4-piperidin-1-yl-butyl)-urea

Prepared via Suzuki Coupling procedure—according to General Method C, to give 37.3 mg (34%)

C₂₀H₂₆ClN₅O Mass (calculated) [387.9]; found [M+H⁺]=388.2

Lc RT=1.56 (10 min method), 100%

NMR (400 MHz, CD₃OD): 1.57-1.69 (4H, m); 1.73-1.89 (6H, m); 3.07-3.12 (2H m), 3.26-3.30 (6H, m), 7.53 (1H, d, J=8.40 Hz), 7.84 (1H, d, J=8.70 Hz), 8.02 (1H, dd, J=2.62 Hz, J=8.70 Hz), 8.35 (1H, dd, J=2.54 Hz, J=8.40 Hz), 8.55 (1H, s), 8.69 (1H, d, J=2.62 Hz), 8.93 (1H, d, J=2.54 Hz).

### Example 21

### 1-[6-(2,4-Dimethoxy-pyrimidin-5-yl)-pyridin-3-yl]-3-(4-piperidin-1-yl-butyl)-urea

Prepared via Suzuki Coupling procedure—according to General Method C, to give 67.1 mg (58%)

C₂₁H₃₀N₆O₃ Mass (calculated) [414.51]; found [M+H⁺]=388.2

Lc RT=0.24 and 1.33 (double peak, 10 min method), 100%

NMR (400 MHz, CD₃OD): 1.57-1.69 (4H, m); 1.73-1.89 (6H, m); 3.07-3.12 (2H, m), 3.26-3.30 (6H, m), 4.05 (3H, s), 4.10 (3H, s), 7.79 (1H, d, J=8.70 Hz), 7.94 (1H, dd, J=2.65 Hz, J=8.71 Hz), 8.53 (1H, s), 8.64 (1H, d, J=2.63 Hz), 8.67 (1H, s).

### Example 22

### 1-(6-Benzofuran-2-yl-pyridin-3-yl)-3-(4-piperidin-1-yl-butyl)-urea

Prepared via Suzuki Coupling procedure—according to General Method C, to give 52.0 mg (47%)

C₂₃H₂₈N₄O₂ Mass (calculated) [392.51]; found [M+H⁺]=393

Lc RT=2.03 (10 min method), 100%

NMR (400 MHz, CD₃OD): 1.31-1.49 (10H, m); 2.20-2.34 (6H, m); 3.05-3.12 (2H, m), 6.42 (1H, s), 7.24 (1H, t, J=7.44 Hz), 7.31 (1H, t, J=7.65 Hz), 7.34 (1H, s), 7.60 (1H, d, J=8.08 Hz), 7.65 (1H, d, J=7.18 Hz), 7.81 (1H, d, J=8.66 Hz), 8.04 (1H, dd, J=2.52 Hz, J=8.66), 8.60 (1H, d, J=2.52 Hz), 8.89 (1H, s).

### Example 23

### Step 1: Preparation of 4-(piperidin-1-yl)butanenitrile

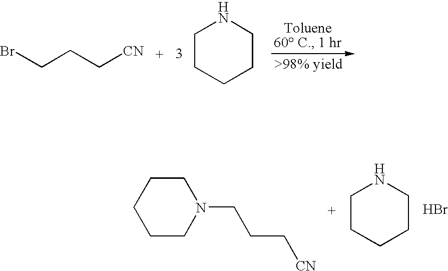

Piperidine (3 equiv.) and toluene (5.5 volumes based on 4-bromobutanenitrile weight) were mixed and heated to 55-60° C. To this mixture, 4-bromobutanenitrile (1 equiv.) was added slowly while keeping temperature below 80° C. Note the addition is exothermic and the exotherm is addition-controlled. A white solid was observed to crystallize during the addition (piperidine hydrobromide). After complete addition, the reaction was stirred at 55-60° C. for an additional 1 hour. Progress of the reaction was monitored by ¹H NMR in CDCl₃ by sampling from the slurry, filtering, and evaporating solvent. After reaction is finished, the mixture was cooled to 10-20° C. and stirred for 1 hour. Solids were removed by filtration, and the cake washed with toluene (2×1.5 volumes based on 4-bromobutanenitrile weight). The combined filtrates were evaporated under vacuum (15-50 mmHg) at 40-50° C. until 120-200% of the theoretical weight was obtained. The solution was again filtered to remove remaining piperidine hydrobromide solids. The filtrate was then further evaporated under vacuum (15-50 mmHg) at 40-50° C. until no more solvent distilled. ¹H NMR in CDCl₃ was done to quantify residual amount of toluene for correction in next step and to verify that there was no residual piperidine present.

### Step 2: Preparation of 4-(piperidin-1-yl)butan-1-amine

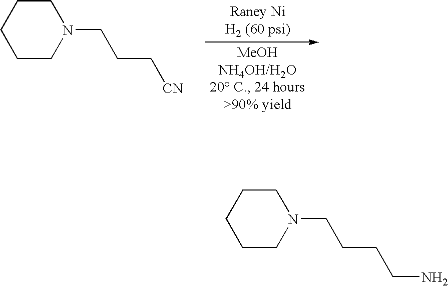

To a pressure reactor was added Raney nickel (30% wt. based on 4-(piperidin-1-yl)butanenitrile weight), a solution of 4-(piperidin-1-yl)butanenitrile (1 equiv.) in MeOH (7.5 volumes based on 4-(piperidin-1-yl)butanenitrile weight), and NH₄OH 28% in H₂O (2.5 volumes based on 4-(piperidin-1-yl)butanenitrile weight). The reactor was purged with N₂ followed by H₂. The reactor was pressurized to 60 psi with H₂ and stirred vigorously for at least 18 hours (until H₂ uptake ceased). The reaction was monitored by TLC (eluent: MeOH/CH₂Cl₂/NH₄OH 2/8/0.5; KMnO₄ stain), and resubjected to the prior step (60 psi H₂) if incomplete. The catalyst was removed by filtration, and the catalyst and reactor were rinsed with methanol (2 volumes based on 4-(piperidin-1-yl)butanenitrile weight). The solvent was distilled under vacuum (15-50 mmHg) at 40-50° C. until no more solvent distilled. The product was distilled under vacuum (10-15 mmHg) at 100-115° C. to give a colorless liquid.

### Step 3 and Step 4

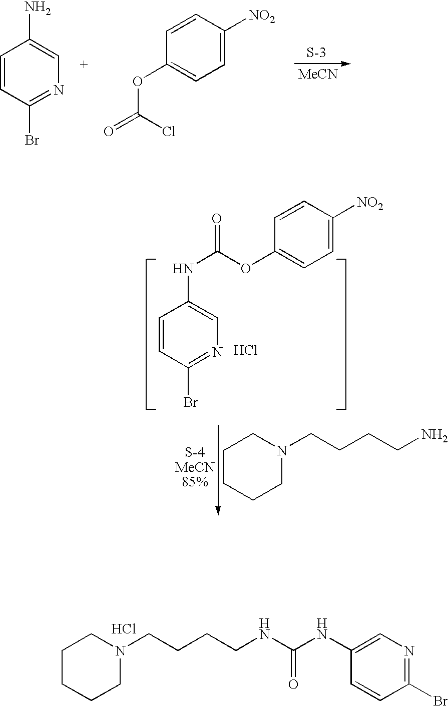

A mixture of 3-Amino-6-bromopyridine (600 g, 3.47 mol) in MeCN (20 L) was prepared in a reactor. 4-Nitrophenyl chloroformate (760 g, 3.77 mol, 1.1 eq) in MeCN (3.8 L, 5×) was added to the reactor over 60 min while maintaining the temperature at 30-40° C. The addition vessel and reactor were rinsed with MeCN (2 L), and the reactor contents were stirred for 2 hours, monitoring reaction progress by HPLC. 1-Piperidinebutanamine (540 g, 3.46 mol, 1 equiv.) in MeCN (1.1 L, 2×) was added to the reactor over 30 min at 30-40° C., and the addition vessel and reactor rinsed with MeCN (200 mL). The reaction was stirred at room temperature for 1 h and monitored by HPLC. Upon completion, the reactor contents were filtered and washed with MeCN (2×1 L). The solids were dried at 40° C. under vacuum for 12 h to yield 1.1 kg (81.7% yield, 98.3% pure).

### Step 5

### Preparation of 1-(6-(4-fluorophenyl)pyridin-3-yl)-3-(4-(piperidin-1-yl)butyl)urea

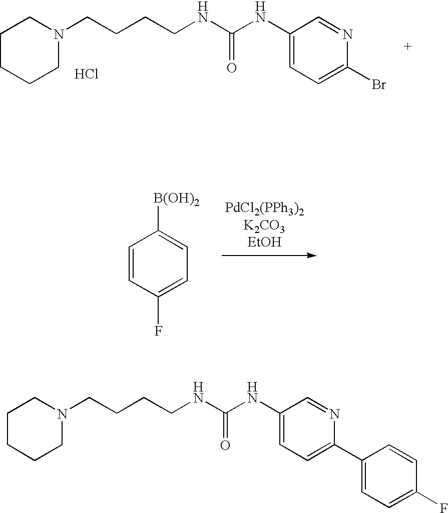

To a reactor, 1-(6-bromopyridin-3-yl)-3-(4-(piperidin-1-yl)butyl)urea hydrochloride 500.0 g, 1.276 mole, 1.00 equiv.), 4-fluorophenylboronic acid (179.0 g, 1.279 mole, 1.00 equiv.), K₂CO₃ powder (353.0 g, 2.554 moles, 2.00 equiv.), PdCl₂(PPh₃)₂ (0.27 g, 0.385 mmol, 0.0003 equiv.) and EtOH ASDQ #7 (7500 mL, 15 vol.), were added. A light gas evolution was observed. The reactor was purged 3 times with N₂ (vacuum 200-250 mmHg). The resulting yellow mixture was heated to reflux (ca. 80° C.) and the progress of the reaction monitored by HPLC. The reaction was considered complete when ≦5% remained. The reaction mixture was distilled to a final volume of 4500 mL (9 vol.), and the resulting mixture cooled to 50-60° C. The mixture was filtered over a Buchner (polypropylene filter). The reactor and filter cake were rinsed with EtOH ASDQ #7 (750 mL, 1.5 vol.), and the filtrate was charged back into the reactor and rinsed using EtOH ASDQ #7 (400 mL, 0.8 vol.). The total volume was between 10 and 10.5 volumes.

The temperature was adjusted to 50-60° C. Over 60 minutes, a solution of NH₄OH 28% (893 mL, 1.8 vol.) in water (4107 mL, 8.2 vol.) was added. Crystallization was observed after the addition of 7.5 to 8 volumes of the NH₄OH solution. The yellow mixture was cooled to 20° C. over 1 hour, then stirred at 20° C. for at least 1 hour. The solids were collected over a Buchner on a polypropylene filter, and the reactor and cake were washed with water (2×1500 mL, 2×3 vol.). The cake was then washed with heptane (1500 mL), and the solids were dried under high vacuum at 50° C. until constant weight was observed, resulting in 434.7 g of 1-(6-(4-fluorophenyl)pyridin-3-yl)-3-(4-(piperidin-1-yl)butyl)urea (92.0% yield) as an off-white solid.

### Step 6

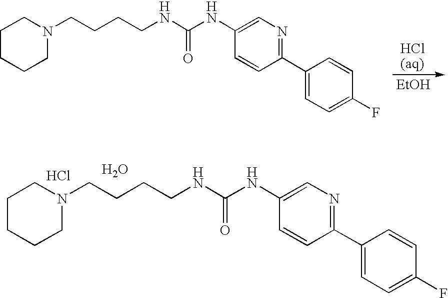

1-(6-(4-fluorophenyl)pyridin-3-yl)-3-(4-(piperidin-1-yl)butyl)urea (700 g, 1.89 mol) was mixed with EtOH (1J1) (3L), and the resulting mixture was heated to 60° C. The mixture was filtered to clarify and rinsed with EtOH (1J1) (0.5 L). Water (140 mL) was added and the temperature adjusted to 30-40° C. To this was added an HCl (20 Be) solution (200 mL, 2.0 mol, 1.06 equiv.) (note that addition is exothermic), and the resulting mixture was stirred for 30 min. Crystal seeds were added, and the resulting mixture stirred for 30 min. at 30-40° C. The mixture was cooled to 0-5° C. and stirred for 1 h. The solids were filtered and dried at room temperature under vacuum for 18 h.

### Example 24

Cloning of Alpha7 Nicotinic Acetylcholine Receptor and Generation of Stable Recombinant Alpha7 nAChR Expressing Cell Lines

Full length cDNAs encoding the alpha7 nicotinic acetylcholine receptor were cloned from a rat brain cDNA library using standard molecular biology techniques. Rat GH4C1 cells were then transfected with the rat receptor, cloned and analyzed for functional alpha7 nicotinic receptor expression employing a FLIPR assay to measure changes in intracellular calcium concentrations. Cell clones showing the highest calcium-mediated fluorescence signals upon agonist (nicotine) application were further subcloned and subsequently stained with Texas red-labelled a-bungarotoxin (BgTX) to analyse the level and homogeneity of alpha7 nicotinic acetylcholine receptor expression using confocal microscopy. Three cell lines were then expanded and one characterised pharmacologically (see Table 4 below) prior to its subsequent use for compound screening.

TABLE 4
Pharmacological characterisation of alpha7 nAChR stably
expressed in GH4C1 cells using the functional FLIPR assay

| Compound | EC₅₀ [microM] |
|---|---|
| Acetylcholine | 3.05 ± 0.08 (n = 4) |
| Choline | 24.22 ± 8.30 (n = 2) |
| Cytisine | 1.21 ± 0.13 (n = 5) |
| DMPP | 0.98 ± 0.47 (n = 6) |
| Epibatidine | 0.012 ± 0.002 (n = 7) |
| Nicotine | 1.03 ± 0.26 (n = 22) |

Development of a Functional FLIPR Assay for Primary Screening

A robust functional FLIPR assay (Z′=0.68) employing the stable recombinant GH4C1 cell line was developed to screen the alpha7 nicotinic acetylcholine receptor. The FLIPR system allows the measurements of real time Ca²⁺-concentration changes in living cells using a Ca²⁺ sensitive fluorescence dye (such as Fluo4). This instrument enables the screening for agonists and antagonists for alpha 7 nAChR channels stably expressed in GH4C1 cells.

Cell Culture

GH4C1 cells stably transfected with rat-alpha7-nAChR (see above) were used. These cells are poorly adherent and therefore pretreatment of flasks and plates with poly-D-lysine was carried out. Cells are grown in 150 cm² T-flasks, filled with 30ml of medium at 37° C. and 5% CO₂.

Data Analysis

EC₅₀ and IC₅₀ values were calculated using the IDBS XLfit4.1 software package employing a sigmoidal concentration-response (variable slope) equation:

Y=Bottom+((Top−Bottom)/(1+((EC₅₀/X)̂HillSlope))

Assay Validation

The functional FLIPR assay was validated with the alpha7 nAChR agonists nicotine, cytisine, DMPP, epibatidine, choline and acetylcholine. Concentration-response curves were obtained in the concentration range from 0.001 to 30 microM. The resulting EC₅₀ values are listed in Table 5 and the obtained rank order of agonists is in agreement with published data (Quik et al., 1997, Mol. Pharmacol., 51, 499-506).

The assay was further validated with the specific alpha7 nAChR antagonist MLA (methyllycaconitine), which was used in the concentration range between 1 microM to 0.01 nM, together with a competing nicotine concentration of 10 microM. The IC₅₀ value was calculated as 1.31±0.43 nM in nine independent experiments.

Development of Functional FLIPR Assays for Selectivity Testing

Functional FLIPR assays were developed in order to test the selectivity of compounds against the alpha1 (muscular) and alpha3 (ganglionic) nACh receptors and the structurally related 5-HT3 receptor. For determination of activity at alpha1 receptors natively expressed in the rhabdomyosarcoma derived TE 671 cell line an assay employing membrane potential sensitive dyes was used, whereas alpha3 selectivity was determined by a calcium-monitoring assays using the native SH-SY5Y cell line. In order to test selectivity against the 5-HT3 receptor, a recombinant cell line was constructed expressing the human 5-HT3A receptor in HEK 293 cells and a calcium-monitoring FLIPR assay employed.

Screening of Compounds

Certain compounds of the present disclosure were tested using the functional FLIPR primary screening assay employing the stable recombinant GH4C1 cell line expressing the alpha7 nAChR. Hits identified were validated further by generation of concentration-response curves. See Table 5, below.

TABLE 5

| | Alpha 7 | Alpha 3 | CYP2D6 | CYP3A4 | CYP2C9 |
|---|---|---|---|---|---|
| | EC₅₀ | EC₅₀ | % | % | % |
| Structure | (μM) | (μM) | inhibition | inhibition | inhibition |
| 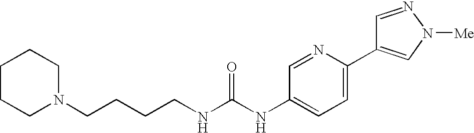 | 0.45 | >30 μM | 1 | −4 | 3 |
| 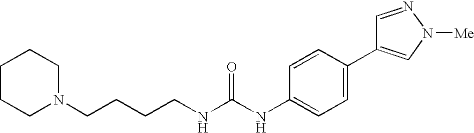 | 0.13 | 5.04 | 45 | 68 | 9 |
| 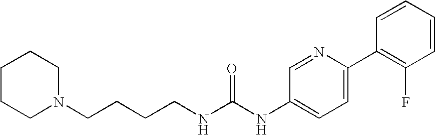 | 0.20 | 5.55 | 14 | −3 | −10 |
| 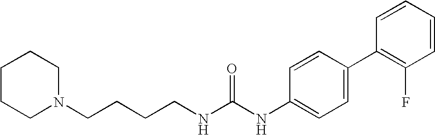 | 0.19 | 1.50 | 75 | −17 | −1 |
| 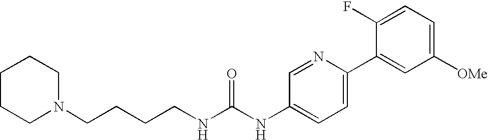 | 0.08 | 4.34 | 18 | 43 | 10 |
| 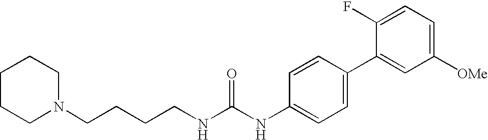 | 0.10 | 2.32 | 83 | 51 | −26 |
| 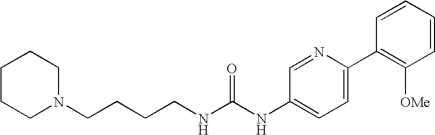 | 0.26 | 3.86 | 8 | 8 | 6 |
| 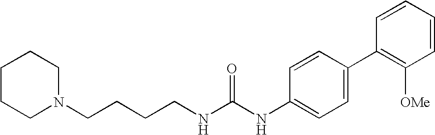 | 0.17 | 1.08 | 38 | 5 | −2 |
| 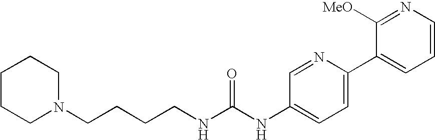 | 0.20 | 15.68 | 8 | −4 | −12 |
| 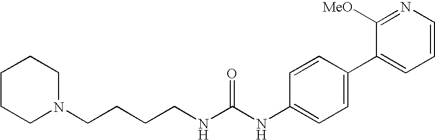 | 0.06 | 5.67 | 8 | −21 | −36 |
| 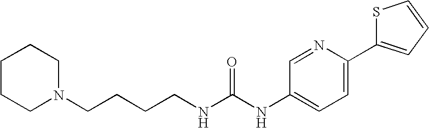 | 0.10 | 8.43 | 7 | 2 | 2 |
| 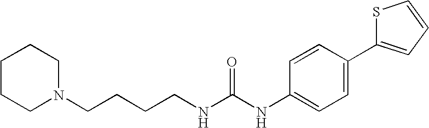 | 0.19 | 3.56 | 69 | 24 | −5 |
| 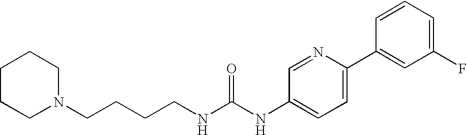 | 0.18 | N/A | 10 | 26 | 0 |
| 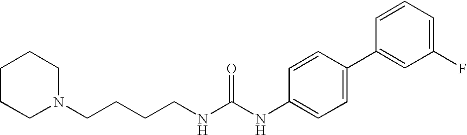 | 0.11 | 1.78 | 62 | −21 | −12 |
| 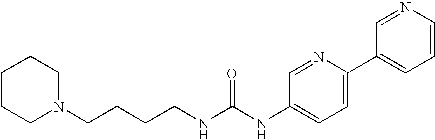 | 0.59 | 9.88 | 22 | 0 | −3 |
| 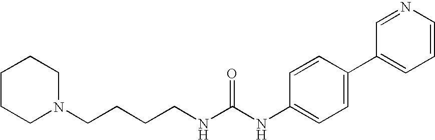 | 0.10 | 3.61 | 72 | 2 | −22 |
| 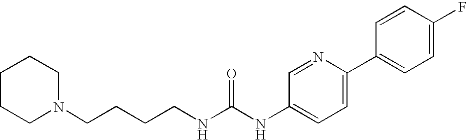 | 0.03 | 9.53 | 15 | −25 | −28 |
| 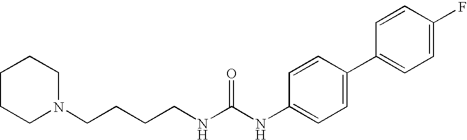 | 0.22 | 1.73 | 65 | −13 | −16 |
| 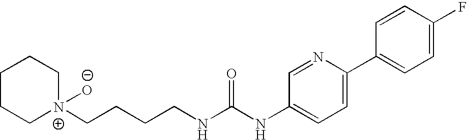 | 0.46 | 9.8 | N/A | N/A | N/A |
| 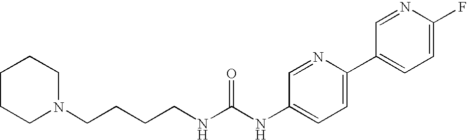 | 0.72 | N/A | 8 | −19 | −10 |
| 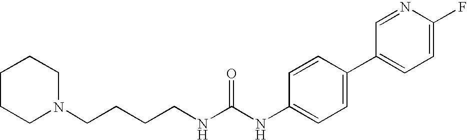 | 0.13 | 1.21 | 21 | −22 | −18 |
| 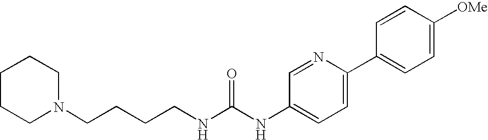 | 0.26 | 4.12 | 54 | −8 | −15 |
| 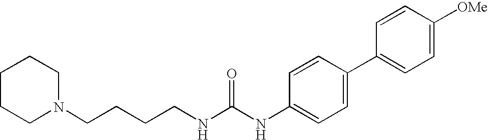 | 0.22 | 1.77 | 97 | 32 | −14 |
| 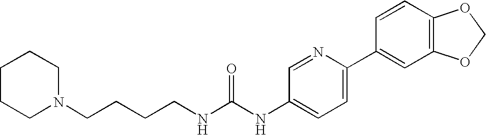 | 0.14 | N/A | 79 | 65 | 6 |
| 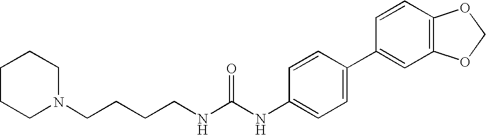 | 0.10 | 1.72 | 97 | 74 | 0 |
| 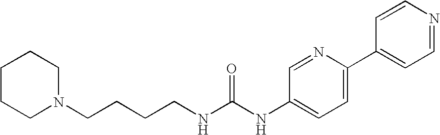 | 0.40 | N/A | 12 | −1 | 1 |
| 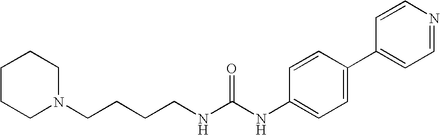 | 0.18 | 2.95 | 48 | 17 | 6 |
| 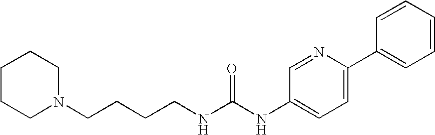 | 0.09 | 10.07 | 6 | −21 | −4 |
| 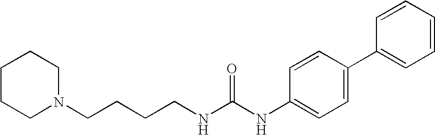 | 0.31 | 2.84 | 64 | 51 | 2 |

### Example 25

### Solid forms of 1-(6-(4-fluorophenyl)pyridin-3-yl)-3-(4-(piperidin-1-yl)butyl)urea hydrochloride

### X-Ray Powder Diffraction Data

X-Ray data were acquired using an X-ray powder diffractometer (Bruker-axs, model D8 advance, Vantec-1 detector) having the following parameters: voltage 40 kV, current 40.0 mA, scan range (2θ) 5 to 30°, total scan time 6 minutes, with a Ni filter. The relative intensities of the peaks can vary, depending upon the sample preparation technique, the sample mounting procedure and the particular instrument employed. Moreover, instrument variation and other factors can often affect the 2-theta values. Therefore, the peak assignments of diffraction patterns can vary by plus or minus about 0.2 degrees 2-theta. Accordingly, as used herein, the term “about” when referencing a 2-theta value, indicates that the value is ±0.2 degrees 2-theta.

### Differential Scanning Calorimetry Data

Differential scanning calorimetry data were collected using a DSC (TA instrument, model Q1000) under the following parameters: 50 mL/min purge gas (N2), scan range 37 to 300° C., scan rate 10° C./min. it is known that the temperatures observed will depend upon the rate of temperature change as well as sample preparation technique and the particular instrument employed. Thus, the values reported herein relating to DSC thermograms can vary by plus or minus about 4° C.

### Thermogravimetric Analysis Data

Thermogravimetric analysis data were collected using a TGA instrument (Mettler Toledo, model TGA/SDTA 851e) under the following parameters: 40 mL/min purge gas(N2); scan range 30 to 300° C., scan rate 20° C./min.

### Monohydrate

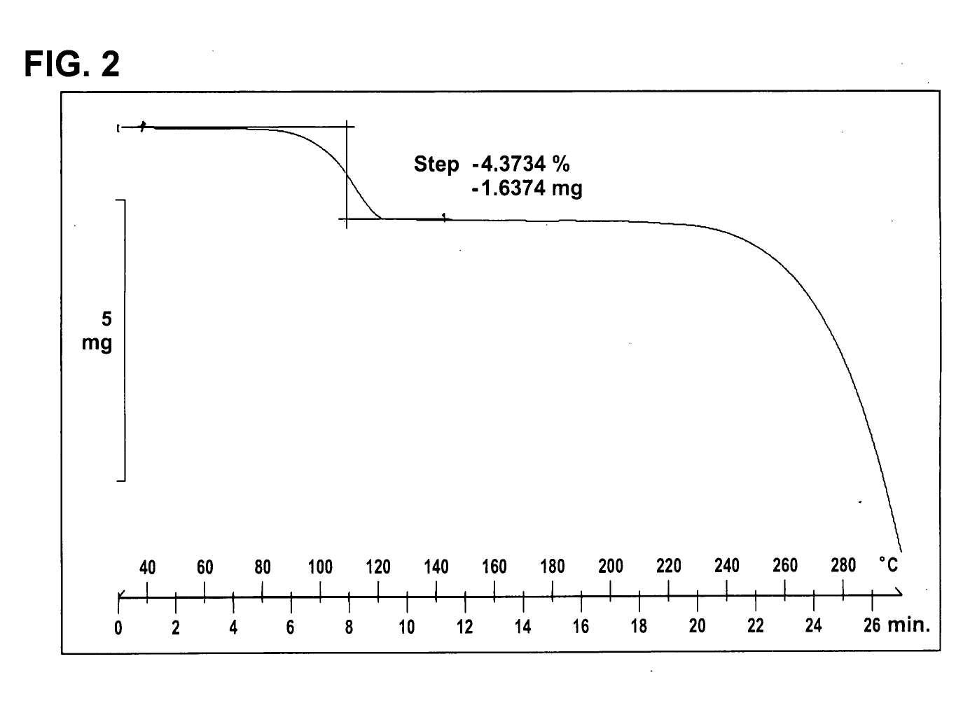

1-(6-(4-Fluorophenyl)pyridin-3-yl)-3-(4-(piperidin-1-yl)butyl)urea (2:3 g of the free base) was added to a multimax reactor. 5 volumes of ethanol (15 ml) were added. The temperature of the reactor was increased to 50° C. and solids dissolved. Water (0.45 ml) was added. HCl (1.05 equivalent moles of 30% HCl aqueous solution) was added. The temperature of the solution was reduced to 35° C. Seeds of the monohydrate form were added to initiate nucleation. The suspension was stirred for 30 min and more crystallization was observed. The suspension temperature was decreased to 0° C. over 30 minutes then stirred over night to maximize the recovery. The solids were dried at room temperature under vacuum. The final crystalline form was characterized by XRD as the monohydrate, KF=4.28%, recovery=78%, HCl content=8.7%. The XRD, TGA, and the DSC of this crystalline form are shown in FIGS. 1, 2, and 3. The theoretical water content of the monohydrate is 4.28%. The analysis for water content by KF showed 4.2%-4.4% thus confirming the monohydrate form. The DSC scan shows two endotherms, one in the range of 70-120° C., and a second one at an onset temperature of around 218° C. The first endotherm is associated with the dehydration of the monohydrate and the second indicates the melting of the dehydrated hydrate.

### Anhydrous

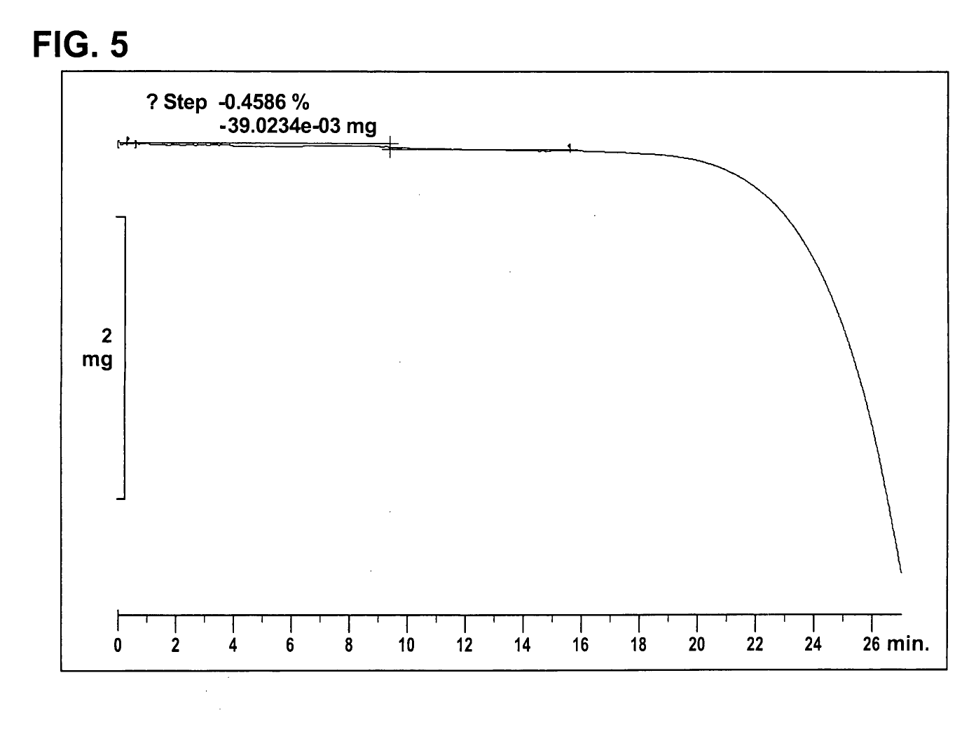

1-(6-(4-Fluorophenyl)pyridin-3-yl)-3-(4-(piperidin-1-yl)butyl)urea (100 mg of the free base) was added to an HPLC vial. 10 volumes (0.5 ml) ethanol were added to the vial. The temperature of the vial was increased to 50° C. on Radley Carousel, while the slurry was being stirred by a small magnet. In a different small vial, 1 equivalent mole of HCl (in the form of HCl solution) was added to 0.15 ml ethanol. The ethanol-acid solution was added to the free base solution. The solution temperature was decreased to RT in 1 hr, and stirred over night. The suspension was filtered and dried in oven under vacuum. The recovery was 73%. The resulting crystalline form was anhydrous having an HCl content of 8.9%. The XRD, TGA, and the DSC scans of the anhydrous form are depicted in FIGS. 4, 5, and 6, respectively. The anhydrous form melts at an onset temperature of around 214° C.

1. Compound I-4: 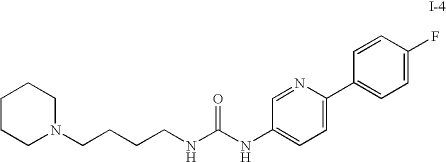
or a pharmaceutically acceptable salt thereof.

2. The compound according to claim 1, as the hydrochloride salt.

3. A crystalline form of the compound according to claim 2.

4. The form according to claim 3, wherein the form is compound I-4 hydrochloride monohydrate.

5. The form according to claim 3, characterized by two endotherms, a first endotherm in the range of 70-120° C., and a second endotherm at an onset temperature of around 218° C.

6. The form according to claim 3, characterized by an XRD pattern substantially similar to that depicted in FIG. 1.

7. The form according to claim 3, characterized in that the form has at least one peak in its XRD pattern selected from about 12.5, 14.2, 19.2, 23.8, or 25.8 degrees 2-theta.

8. The form according to claim 3, characterized in that the form has at least two peaks in its XRD pattern selected from about 12.5, 14.2, 19.2, 23.8, or 25.8 degrees 2-theta.

9. The form according to claim 3, characterized in that the form has substantially all of the following peaks in its XRD pattern:

| Angle | d value | Intensity % |
|---|---|---|
| 2-Theta ° | Angstrom | % |
| 6.4 | 13.9 | 19.1 |
| 10.7 | 8.3 | 8.0 |
| 11.9 | 7.4 | 21.3 |
| 12.5 | 7.1 | 49.1 |
| 12.7 | 7.0 | 42.3 |
| 14.2 | 6.3 | 81.8 |
| 14.4 | 6.1 | 8.6 |
| 15.9 | 5.6 | 24.1 |
| 17.2 | 5.2 | 28.7 |
| 18.5 | 4.8 | 30.2 |
| 18.9 | 4.7 | 76.5 |
| 19.2 | 4.6 | 99.4 |
| 19.3 | 4.6 | 60.2 |
| 19.6 | 4.5 | 14.8 |
| 19.9 | 4.5 | 100.0 |
| 20.2 | 4.4 | 69.8 |
| 21.0 | 4.2 | 54.3 |
| 21.5 | 4.1 | 30.9 |
| 23.0 | 3.9 | 25.6 |
| 23.5 | 3.8 | 24.7 |
| 23.8 | 3.7 | 75.6 |
| 24.2 | 3.7 | 62.7 |
| 24.3 | 3.7 | 58.6 |
| 25.5 | 3.5 | 23.1 |
| 25.8 | 3.5 | 65.1 |
| 26.1 | 3.4 | 14.5 |
| 26.7 | 3.3 | 12.0 |
| 26.9 | 3.3 | 15.4 |
| 27.8 | 3.2 | 9.6 |
| 28.6 | 3.1 | 29.0 |
| 29.0 | 3.1 | 12.7 |
| 28.3 | 3.1 | 9.9 |
| 29.6 | 3.0 | 9.6 |

10. The form according to claim 3, wherein the form is compound I-4 hydrochloride anhydrous.

11. The form according to claim 3, characterized by a melting point of about 214° C.

12. The form according to claim 3, characterized by an XRD pattern substantially similar to that depicted in FIG. 4.

13. The form according to claim 3, characterized in that the form has at least one peak in its XRD pattern selected from about 10.0, 11.3, 12.3, 14.9, 17.0, 17.5, 17.9, 19.0, 21.5, or 22.8 degrees 2-theta.

14. The form according to claim 13, characterized in that the form has at least two peaks in its XRD pattern selected from about 10.0, 11.3, 12.3, 14.9, 17.0, 17.5, 17.9, 19.0, 21.5, or 22.8 degrees 2-theta.

15. The form according to claim 3, characterized in that the form has substantially all of the following peaks in its XRD pattern:

| Angle | d value | Intensity % |
|---|---|---|
| 2-Theta ° | Angstrom | % |
| 29.8 | 3.0 | 39.0 |
| 28.5 | 3.1 | 30.6 |
| 27.2 | 3.3 | 30.0 |
| 26.1 | 3.4 | 29.3 |
| 25.4 | 3.5 | 37.7 |
| 25.2 | 3.5 | 37.8 |
| 24.1 | 3.7 | 30.0 |
| 22.8 | 3.9 | 33.4 |
| 22.1 | 4.0 | 54.5 |
| 21.5 | 4.1 | 100.0 |
| 21.2 | 4.2 | 43.8 |
| 20.1 | 4.4 | 31.3 |
| 19.9 | 4.5 | 35.2 |
| 19.7 | 4.5 | 31.0 |
| 19.0 | 4.7 | 57.3 |
| 17.9 | 4.9 | 34.6 |
| 17.5 | 5.1 | 30.5 |
| 17.2 | 5.1 | 33.9 |
| 17.0 | 5.2 | 31.9 |
| 16.2 | 5.5 | 29.7 |
| 15.8 | 5.6 | 28.0 |
| 14.9 | 5.9 | 33.6 |
| 14.4 | 6.2 | 29.3 |
| 13.5 | 6.6 | 29.9 |
| 12.8 | 6.9 | 41.2 |
| 12.3 | 7.2 | 43.8 |
| 11.3 | 7.8 | 30.3 |
| 10.0 | 8.9 | 29.2 |

16. A pharmaceutical composition comprising a compound according to claim 1 and a pharmaceutically acceptable excipient.

17. A method for the treatment neurological, neurodegenerative, psychiatric, cognitive, immunological, inflammatory, metabolic, addiction, nociceptive, and sexual disorders, the method comprising administering to a subject in need thereof a therapeutically effective amount of a compound according to claim 1.

19. The method according to claim 17, wherein the disorder is senile dementia, attention deficit disorders, Alzheimer's disease, or schizophrenia.

20. A method for the prevention or treatment of diseases, conditions or dysfunctions involving the alpha 7 nAChR, the method comprising administering to a subject in need thereof a therapeutically effective amount of a compound according to claim 1.

21. The method according to claim 20, wherein the disease, condition, or dysfunction is senile dementia, attention deficit disorders, Alzheimer's disease, or schizophrenia.
